(19) United States
(12) Patent Application Publication
(10) Pub. No.: US 2002/0031844 A1
(43) Pub. Date: Mar. 14, 2002

(54) Biochemical analysis apparatus

(75) Inventors: Akihiro Komatsu, Kanagawa (JP); Fumio Sugaya, Kanagawa (JP); Yoichi Endo, Kanagawa (JP); Tomoyuki Takiue, Kanagawa (JP)

Correspondence Address: SUGHRUE, MION, ZINN, MACPEAK & SEAS, PLLC, 2100 Pennsylvania Avenue, N.W., Washington, DC, 20037-3202, US

(73) Assignee: FUJI PHOTO FILM CO., LTD.

(21) Appl. No.: 09/950,882

(22) Filed: Sep. 13, 2001

(30) Foreign Application Priority Data
Sep. 13, 2000 (JP) 278781/2000
Sep. 13, 2000 (JP) 278782/2000

(51) Int. Cl. C12M 1/34; C12Q 1/68; G01N 33/543

(52) U.S. Cl. 436/518; 435/287.2; 435/6

(57) ABSTRACT

Disclosed herein is a biochemical analysis apparatus for measuring the optical density and ionic activity of a biochemical substance. The biochemical analysis apparatus is equipped with first and second dry analysis chips, different in method of measurement, which have inspection matter dropped thereon, a first incubator for housing the first dry analysis chip and incubating the first dry analysis chip at a first predetermined temperature, and a first measurement section provided in the first incubator. The biochemical analysis apparatus is further equipped with a second incubator for housing the second dry analysis chip and incubating the second dry analysis chip at a second predetermined temperature, a second measurement section provided in the second incubator, and a conveyance section for conveying the first and second dry analysis chips to the first and second incubators through first and second conveying paths.

### BACKGROUND OF THE INVENTION

1. Field of the Invention

The present invention relates to a biochemical analysis apparatus for detecting the density, ionic activity, etc., of a predetermined biochemical substance contained in inspection matter such as blood, urine, etc., by employing different types of dry analysis chips on which the inspection matter is dropped.

2. Description of the Related Art

A colorimetric-type dry analysis chip has been put to practical use. If only a small quantity of inspection matter is dropped and supplied to the dry analysis chip, a specific chemical component or concrete component in the inspection matter can be quantitatively analyzed from the color reaction. In a biochemical analysis apparatus, which makes a quantitative analysis of a chemical component contained in inspection matter by employing such a dry analysis chip, the inspection matter is first dropped on the dry analysis chip. Then, the dry analysis chip is incubated for a predetermined time by an incubator so that a color reaction (dye generation reaction) occurs. Next, the dry analysis chip is irradiated with measuring light which contains a wavelength previously selected according to a combination of a predetermined biochemical substance in the inspection matter and a reagent contained in the dry analysis chip, and the optical density is measured. Based on the measured optical density, the substance density of the predetermined biochemical substance in the inspection matter is obtained by employing an analytical curve which represents a corresponding relationship between the optical density previously obtained and the substance density of the predetermined biochemical substance in the inspection matter.

An electrolytic-type dry analysis chip has also been put to practical use. It is used to measure the ionic activity of a specific ion contained in the inspection matter. The dry analysis chip for measuring ionic activity has at least one ion selecting electrode pair for generating an electric potential which corresponds to the ionic activity of a specific ion, and a porous bridge disposed to connect the ion selecting electrodes. If a reference solution with known ionic activity and inspection matter with unknown ionic activity are respectively dropped and supplied to the ion selecting electrodes, and both solutions are electrically connected through the porous bridge, a potential difference is generated between both electrodes in accordance with the difference between the ionic activities of the ions present between the reference solution and the inspection matter. If the potential difference is measured, the ionic activity of a specific ion in the inspection matter can be obtained based on a previously calculated analytical curve (whose principle is based on Nernst's equation).

The biochemical analysis apparatus, for measuring ionic activity by employing such a dry analysis chip, is required to have the function of performing the dropping and supply of a reference solution and inspection matter and a measurement of potential difference. After the dropping of the reference solution and the inspection matter, the dry analysis chip is sent to a potential-difference measuring section. In the measuring section, potential measuring probes are respectively contacted with both electrodes and measure the potential difference between the electrodes.

To remove the inconvenience of making different kinds of measurements with different biochemical analysis apparatuses, there has been proposed a biochemical analysis apparatus equipped with a plurality of measurement means.

One such apparatus is disclosed in Japanese Unexamined Patent Publication No. 2(1990)-44034. In this biochemical analysis apparatus, two different dry analysis chips are taken out from different cartridges housing dry analysis chips and are inserted into a single incubator. Thereafter, the electrolytic-type dry analysis chip is taken out from the incubator and sent to a potential measuring unit, in which it is measured. Also, the colorimetric-type dry analysis chip is measured with a photometric unit, while it is being held in the incubator.

Another apparatus is disclosed in Japanese Unexamined Patent Publication No. 11(1999)-211730. In this biochemical analysis apparatus, different types of dry analysis chips are housed in a single cartridge. After dry analysis chips have been taken out one by one, they are inserted into a single incubator. The electrolytic-type dry analysis chip, as with the colorimetric-type dry analysis chip, is measured with a potential-difference measuring section provided within the incubator.

However, in the former, colorimetric measurement cannot be performed efficiently, because the portion of the incubator in which calorimetric measurement is performed is occupied during the time that an electrolytic-type dry analysis chip is being inserted in the incubator. In addition, when the incubation temperature of the electrolytic-type dry analysis chip differs from that of the colorimetric-type dry analysis chip, they cannot be simultaneously used. Furthermore, since the incubator is incorporated with a taking-out mechanism for taking out an electrolytic-type dry analysis chip to a potential measuring unit, a reduction in the size of the incubator is difficult and therefore there is a problem that the apparatus will be increased in size and will become structurally complicated.

On the other hand, in the latter, there is no need to take out an electrolytic-type dry analysis chip from the incubator, so the size of the incubator can be reduced. In addition, since an electrolytic-type dry analysis chip is incubated in the potential-difference measuring section, dry analysis chips differing in incubation temperature can be used. However, because rotation of the incubator is stopped during the potential measurement of the electrolytic-type dry analysis chip, there is a problem that a colorimetric measurement of a colorimetric-type dry analysis chip cannot be efficiently made.

### SUMMARY OF THE INVENTION

The present invention has been made in view of the aforementioned circumstances. Accordingly, it is an object of the present invention to provide a biochemical analysis apparatus which is capable of efficiently processing different types of dry analysis chips without increasing the size of the apparatus, particularly the incubator. Another object of the invention is to provide a biochemical analysis apparatus that is capable of efficiently measuring different kinds of dry analysis chips that differ in incubation temperature.

To achieve the objects of the present invention mentioned above, there is provided a first biochemical analysis apparatus comprising (1) first and second dry analysis chips, different in method of measurement, which have a dropped inspection matter; (2) a first incubator for housing the first dry analysis chip and incubating the first dry analysis chip at a first predetermined temperature; (3) first measurement means provided in the first incubator; (4) a second incubator for housing the second dry analysis chip and incubating the second dry analysis chip at a second predetermined temperature; (5) second measurement means provided in the second incubator; and (6) conveyance means for conveying the first and second dry analysis chips to the first and second incubators through first and second conveying paths.

In the first biochemical analysis apparatus of the present invention, it is preferable that the conveyance means comprise a first conveying member for conveying the first and second dry analysis chips to a distributing section and also conveying the first dry analysis chip from the distributing section to the first incubator, and a second conveying member for conveying the second dry analysis chip from the distributing section to the second incubator.

In the first biochemical analysis apparatus, it is preferable that the distributing section comprise a first guide member, which projects from a conveying surface, for guiding the first dry analysis chip to the first incubator, and a second guide member, which projects from the conveying surface, for guiding the second dry analysis chip to the second incubator.

In the first biochemical analysis apparatus, it is preferable that the first and second dry analysis chips after measurement are conveyed beyond the first and second incubators and are discarded.

In the first biochemical analysis apparatus, the first and second dry analysis chips are each provided with a bar code that indicates its type. The bar code is read before dropping of the inspection matter, and according to the type, the dropping, conveyance, incubation, and measurement are performed.

In the first biochemical analysis apparatus, the first dry analysis chip is a colorimetric type dry analysis chip for measuring a substance density of a predetermined biochemical substance contained in the inspection matter by color reaction. The second dry analysis chip is an electrolytic type dry analysis chip for measuring ionic activity of the inspection matter. The first measurement means comprises a color-reaction measuring section for measuring a change in optical density by a color reaction between the predetermined biochemical substance and a reagent. The second measurement means comprises a potential-difference measuring section equipped with probes for measuring a potential difference between the inspection matter and a reference solution which corresponds to the ionic activity. The first incubator has a plurality of chip chambers, and a measurement is made in sequence with the first measurement means. The second incubator has a single chip chamber, and a measurement is made with the second measurement means.

According to the first biochemical analysis apparatus of the present invention, a first dry analysis chip and a second dry analysis chip differing in method of measurement are conveyed to a first incubator and a second incubator and are separately measured with first measurement means and second measurement means. Therefore, different types of measurements can be made at the same time. In addition, the first and second dry analysis chips can be incubated at respective temperatures, so there is no need to wait until one of the two measurements ends. Thus, the first biochemical analysis apparatus of the present invention is capable of efficiently processing and measuring dry analysis chips that differ in method of measurement.

In addition, the first biochemical analysis apparatus is equipped with conveyance means for conveying the first and second dry analysis chips to the first and second incubators through first and second conveying paths. Therefore, the size of each incubator can be reduced, and the size of the apparatus can be reduced because the first and second conveying paths partially overlap each other.

The conveyance means in the first biochemical analysis apparatus of the present invention is constructed of a first conveying member for conveying the first and second dry analysis chips to a distributing section and also conveying the first dry analysis chip from the distributing section to the first incubator, and a second conveying member for conveying the second dry analysis chip from the distributing section to the second incubator. Therefore, the layout and structure of the biochemical analysis apparatus can be further simplified.

If the distributing section of the conveyance means is provided with first and second guide members for guiding the first dry analysis chip to the first and second incubators, switching of the conveying directions of the first and second dry analysis chips can be certainly performed and therefore reliability can be enhanced.

If the first and second dry analysis chips after measurement are conveyed beyond the first and second incubators and are discarded, there is no need to provide a mechanism for discarding analysis chips and therefore the conveying mechanism can be made structurally simpler.

If the first and second dry analysis chips are each provided with a bar code that represents type, and the bar code is read before dropping of inspection matter, the dropping, conveyance, incubation, and measurement can be performed according to the analysis chip type. The sequence of operations can be accurately performed and efficient measurement can be made.

In the first biochemical analysis apparatus, the first dry analysis chip is a colorimetric type dry analysis chip for measuring the density of a substance. The second dry analysis chip is an electrolytic type dry analysis chip for measuring ionic activity. The first measurement means is constructed of a color-reaction measuring section, and the second measurement means is constructed of a potential-difference measuring section. The first incubator has a plurality of chip chambers, and the second incubator has a single chip chamber. Therefore, efficient measurement can be made according to actual measurement and the apparatus can be made compact.

In accordance with the present invention, there is provided a second biochemical analysis apparatus comprising (1) first and second dry analysis chips differing in method of measurement; (2) a dropping section for dropping inspection matter to the first and second dry analysis chips; (3) a first incubator for housing the first dry analysis chip which has the dropped inspection matter and then incubating the first dry analysis chip at a first predetermined temperature; (4) first measurement means provided in the first incubator; (5) a second incubator for housing the second dry analysis chip which has the dropped inspection matter and then incubating the second dry analysis chip at a second predetermined temperature; (6) second measurement means provided in the second incubator; and (7) a distributing section disposed between the dropping section and the first incubator; wherein a passage for conveying the first dry analysis chip to the first incubator, and a chip chamber of the second incubator, are provided in the distributing section so that the passage and the chip chamber can be switched between them; and wherein the second incubator and the second measurement means are disposed in the distributing section.

In the second biochemical analysis apparatus of the present invention, the passage and the chip chamber in the distributing section are provided parallel to each other in a vertical direction with respect to a conveying path and are movable up and down, depending on dry analysis chip type. Also, the passage and the chip chamber in the distributing section may be provided parallel to each other in a lateral direction with respect to a conveying path and are movable in the lateral direction, depending on dry analysis chip type.

In the second biochemical analysis apparatus, the second incubator is movable integrally with movement of the passage and the chip chamber in the distributing section. Also, the second incubator may be fixedly disposed and the chip chamber with the second dry analysis chip housed therein may be movable with respect to the second incubator.

In the second biochemical analysis apparatus, the first and second dry analysis chips are each provided with a bar code that indicates its type. The bar code is read before dropping of the inspection matter, and according to the type, the dropping, conveyance, incubation, and measurement are performed.

In the second biochemical analysis apparatus, as with the first biochemical analysis apparatus, the first dry analysis chip is a calorimetric type dry analysis chip for measuring a substance density of a predetermined biochemical substance contained in the inspection matter by color reaction. The second dry analysis chip is an electrolytic type dry analysis chip for measuring ionic activity of the inspection matter. The first measurement means comprises a color-reaction measuring section for measuring a change in optical density by color reaction between the predetermined biochemical substance and a reagent. The second measurement means comprises a potential-difference measuring section equipped with probes for measuring a potential difference between the inspection matter and a reference solution which corresponds to the ionic activity. The first incubator has a plurality of chip chambers, and a measurement is made in sequence with the first measurement means. The second incubator has a single chip chamber, and when the second dry analysis chip is inserted into the single chip chamber and moved, the probes of the second measurement means are connected electrically with the second dry analysis chip.

According to the second biochemical analysis apparatus of the present invention, as with the first biochemical analysis apparatus, a first dry analysis chip and a second dry analysis chip differing in method of measurement are conveyed to a first incubator and a second incubator and are separately measured with first measurement means and second measurement means. Therefore, different types of measurements can be made at the same time. In addition, the first and second dry analysis chips can be incubated at their respective incubation temperatures, so there is no need to wait until one of the two measurements ends. Thus, the second biochemical analysis apparatus of the present invention is capable of efficiently processing and measuring dry analysis chips that differ in method of measurement.

In the second biochemical analysis apparatus, the first dry analysis chip is inserted into the first incubator through the distributing section, and the second dry analysis chip is inserted into the chip chamber of the second incubator provided in the distributing section. Therefore, the size of each incubator can be reduced, and the size of the apparatus can be reduced because the first and second conveying paths partially overlap each other.

If a bar code provided in the dry analysis chip is read before dropping of inspection matter, as with the first biochemical analysis apparatus, the dropping, conveyance, incubation, and measurement can be performed according to the analysis chip type. The sequence of operations can be accurately performed and efficient measurement can be made.

In the second biochemical analysis apparatus, as with the first biochemical analysis apparatus, the first dry analysis chip is a colorimetric type dry analysis chip for measuring the density of a substance. The second dry analysis chip is an electrolytic type dry analysis chip for measuring ionic activity. The first measurement means is constructed of a color-reaction measuring section, and the second measurement means is constructed of a potential-difference measuring section. The first incubator has a plurality of chip chambers, and the second incubator has a single chip chamber. Therefore, efficient measurement can be made according to actual measurement and the apparatus can be made compact.

If the probes of the second measurement means are contacted electrically with the second analysis chip when the chip chamber of the second incubator with the second dry analysis chip housed therein is moved, the apparatus can be made structurally simpler.

### BRIEF DESCRIPTION OF THE DRAWINGS

The present invention will be described in further detail with reference to the accompanying drawings wherein:

FIG. 1 is a plan view of a biochemical analysis apparatus according to a first embodiment of the present invention;

FIG. 2 is a front sectional view of the chip stand-by section, the dropping section, the distributing section, and the conveyance means shown in FIG. 1;

FIG. 3 is a plan view of the distributing section, the chip press of the distributing section having been removed;

FIG. 4 is a sectional front view showing the distributing section;

FIG. 5 is a sectional front view of the first incubator shown in FIG. 1;

FIG. 6 is a sectional view of the second incubator and the second measurement means shown in FIG. 1;

FIG. 7 is a sectional view of the sample housing section shown in FIG. 1;

FIG. 8 is a plan view of the blood-plasma filtering unit shown in FIG. 1;

FIG. 9 is a sectional front view of the dropping means shown in FIG. 1;

FIG. 10 is a sectional view of the dropping nozzle shown in FIG. 9;

FIG. 11 is a perspective view showing dry analysis chips used in the biochemical analysis apparatus of FIG. 1;

FIG. 12 is a plan view of a biochemical analysis apparatus according to a second embodiment of the present invention;

FIG. 13 is a front sectional view of the chip stand-by section, the dropping section, the distributing section, and the conveyance means shown in FIG. 12;

FIG. 14 is a sectional front view showing the operating state of the distributing section of FIG. 12;

FIG. 15 is a sectional front view of the first incubator shown in FIG. 12;

FIG. 16 is a sectional view of the sample housing section shown in FIG. 12;

FIG. 17 is a plan view of the blood-plasma filtering unit shown in FIG. 12;

FIG. 18 is a sectional front view of the dropping means shown in FIG. 12;

FIG. 19 is a sectional view of the dropping nozzle shown in FIG. 18;

FIG. 20 is a perspective view showing dry analysis chips used in the biochemical analysis apparatus of FIG. 12;

FIG. 21 is a plan view of the distributing section of a biochemical analysis apparatus according to a third embodiment of the present invention; and

FIG. 22 is a plan view of the distributing section of a biochemical analysis apparatus according to a fourth embodiment of the present invention.

### DESCRIPTION OF THE PREFERRED EMBODIMENTS

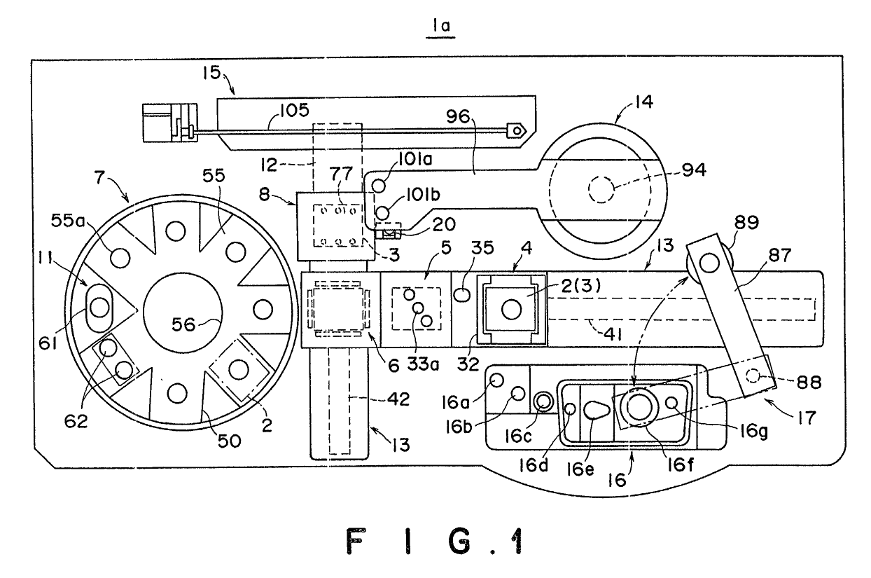

Referring now in greater detail to the drawings and initially to FIG. 1, there is shown a biochemical analysis apparatus 1a in accordance with a first embodiment of the present invention. The biochemical analysis apparatus 1a is equipped with a chip stand-by section (chip housing section) 4 for housing a first dry analysis chip (colorimetric-type dry analysis chip) 2 and a second dry analysis chip (electrolytic-type dry analysis chip) 3 in a mixed state; a dropping section 5, following the chip stand-by section 4, for dropping inspection matter (which is whole blood, serum, urine, blood plasma, etc., but, in this embodiment, only blood plasma will be described) on the dry analysis chips 2, 3; and a distributing section 6, following the dropping section 5, for conveying the second dry analysis chip 3. The biochemical analysis apparatus 1a is also equipped with a first incubator 7, disposed near the front surface of the distributing section 6, for incubating the first dry analysis chip 2 for a predetermined time; a second incubator 8, disposed near the side surface of the distributing section 6, for incubating the second dry analysis chip 3 for a predetermined time; first measurement means 11 for measuring the first dry analysis chip 2 installed in the first incubator 7; and second measurement means 12 for measuring the second dry analysis chip 3 installed in the second incubator 8.

The biochemical analysis apparatus 1a is further equipped with conveyance means 13 for conveying the first dry analysis chip 2 and the second dry analysis chip 3 to the first incubator 7 and the second incubator 8. This conveyance means 13 has the distributing section 6, disposed between the dropping section 5 and the first incubator 7. The conveyance means 13 is equipped with a first conveying member 41 movable linearly from the chip stand-by section 4 to the first incubator 7, and a second conveying member 42 movable from the distributing section 6 to the second incubator 2 in a direction perpendicular to the conveying direction of the first dry analysis chip 2.

The first and second analysis chips 2, 3 are conveyed in sequence from the chip stand-by section 4 to the dropping section 5 by the first conveying member 41 of the conveyance means 13. In the dropping section 5, inspection matter is dropped on the first dry analysis chip 2 by dropping means (sampler) 14, and inspection matter and a reference solution are dropped on the second dry analysis chip 3 by the dropping means 14. Thereafter, the first dry analysis chip 2 with the dropped inspection matter is linearly inserted into the first incubator 7 through the distributing section 6 by the first conveying member 41. The second dry analysis chip 3 is changed in direction at the distributing section 6 by 90° and inserted into the second incubator 8 by the second conveying member 42.

The coloration (optical reflection density) of the first dry analysis chip 2 incubated by the first incubator 7 is measured with the first measurement means 11, and a potential difference in the second dry analysis chip 3 incubated by the second incubator 8 is measured with the second measurement means 12. After the measurements, the first dry analysis chip 2 is further conveyed by the first conveying member 41 and is dropped and discarded through the center portion of the first incubator 7. The second dry analysis chip 3 is further conveyed by the second conveying member 42 and is discarded behind the second incubator 8.

Note that the dropping means 14 fits a nozzle tip 21 (described later) onto the tip end of a dropping nozzle 101a or 101b, then draws in by suction inspection matter (blood plasma), a reference solution, etc., from a sample housing section 16 into the nozzle tip 21, and drops a predetermined quantity of inspection matter on the dry analysis chip 2 or 3. To drop inspection matter, the dropping means 14 is provided with syringe means 15 which draws and expels the inspection matter by the nozzle tip 21. After it is used, the nozzle tip 21 is removed by a nozzle-tip removing member 20 (FIG. 1) and is dropped and discarded downward. Also, a blood filtering unit 17 is disposed near the sample housing section 16 to separate blood plasma from blood.

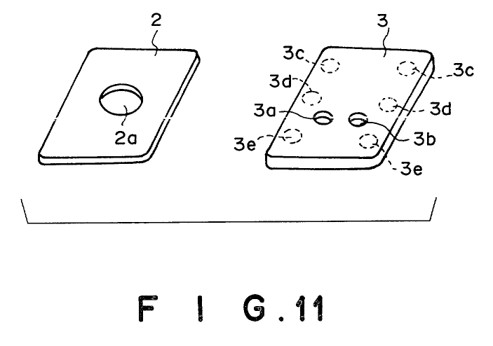

Now, the construction of the first dry analysis chip 2 of the colorimetric type used for measuring the coloration of blood plasma, and the construction of the second dry analysis chip 3 of the electrolytic type used for measuring the ionic activity of blood plasma, will be described with reference to FIG. 11. The first dry analysis chip 2 on the left side is constructed of a rectangular mount within which a layer of regent is disposed. The mount has an inspection-matter receiving bore 2a to which inspection matter is dropped. On the other hand, the second dry analysis chip 3 on the right side is approximately the same in shape as the first dry analysis chip 2, and has an inspection-matter receiving bore 3a to which inspection matter is dropped, and a reference-solution receiving bore 3b to which a reference solution with known ionic activity is dropped. The second dry analysis chip 3 also has three pairs of ion selecting electrodes 3c, 3d, 3e, which are connected electrically with the potential measuring probes of the second measurement means 12 to measure ionic activity. The ion selecting electrode pairs 3c, 3d, 3e have Cl⁻-, K⁺-, and Na⁺-selecting layers, respectively. Also, the bottom surfaces of the first and second dry analysis chips 2, 3 are each provided with a bar code (not shown) indicating information that identifies an inspection item, etc.

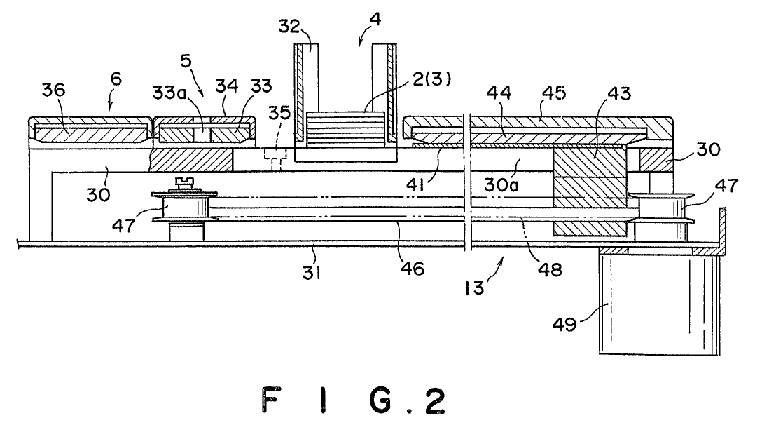

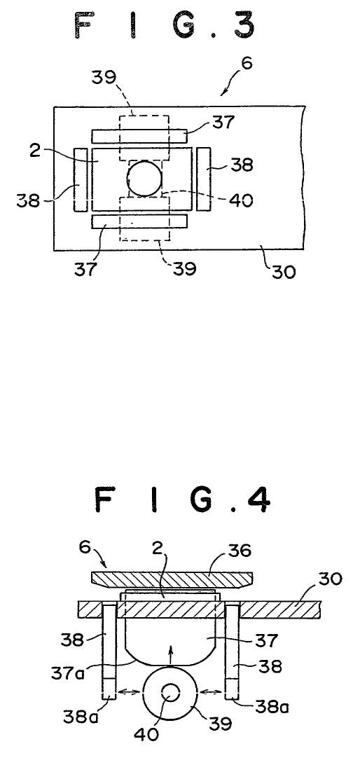

Next, the construction of each section of the biochemical analysis apparatus 1a will be described. Initially, the chip stand-by section 4, the dropping section 5, the distributing section 6, and the conveyance means 13 will be described with reference to FIG. 2. The conveyance means 13 is equipped with a conveying table 30 linearly extending toward the center of the first incubator 7. The conveying table 30 is installed on a flat base 31. The chip stand-by section 4 is disposed in approximately the central portion of the conveying table 30, and the dropping section 5 and the distributing section 6 are disposed on the side of the conveying table 30 closer to the first incubator 7 than the central portion of the conveying table 30.

The chip stand-by section 4 is provided with a chip guide 32. In the chip guide 32, a plurality of first and second dry analysis chips 2, 3 unused are stacked and held in a mixed state. Also, the lowermost analysis chip 2 or 3 in the chip guide 32 is coplanar with the conveying surface of the conveying table 30. The front wall of the chip guide 32 has a front slit through which only a single analysis chip 2 or 3 is passed, while the rear wall has a rear slit into which the first conveying member 41 is inserted. Note that a cartridge with a plurality of dry analysis chips 2, 3 stacked together may be provided in the chip guide 32.

The dropping section 5 is provided with a chip press 33 that has three dropping bores 33a. The chip press 33 is housed within a cover 34 fixedly attached above the conveying table 30. A bar-code reader 35 is interposed between the chip stand-by section 4 and the dropping section 5 in order to read the bar code of the dry analysis chip 2 or 3. The bar-code reader 35 is provided for specifying an inspection item, etc., for controlling the dropping of inspection matter and a reference solution, conveyance, and measurement, and also for detecting the conveying direction, etc., of the dry analysis chip 2 or 3 (e.g., the forward direction, rearward direction, top surface, bottom surface, etc.).

The distributing section 6 is equipped with a chip press 36 disposed above the chip stopping portion of the conveying table 30, as shown in FIGS. 2 and 4. The distributing section 6 is further equipped with a first guide, provided as a paired member (hereinafter referred to as guide pair) 37 which projects from the conveying surface of the conveying table 30 to guide the first dry analysis chip 2 toward the first incubator 7, and a second guide pair 38 which projects from the conveying surface to guide the second dry analysis chip 3 toward the second incubator 8.

The first guide pair 37 is provided in parallel with the conveying direction of the first incubator 7, while the second guide pair 38 is provided perpendicular to the first guide pair 37 and parallel to the direction where the second dry analysis chip 3 is conveyed from the distributing section 6 to the second incubator 8. The first and second guide pairs 37, 38 are urged downward by dead weights or springs, and the bottom surfaces are formed into cam surfaces 37a, 38a. The first and second guide pairs 37, 38 are moved upward by contacting with a roller 39. The roller 39 is mounted on the opposite end portions of a horizontal shaft 40 so that it can abut the cam surfaces 37a, 38a of the first and second guide pairs 37, 38. The horizontal shaft 40 is rotatable through 90° by rotation means (not shown). With rotation of the horizontal shaft 40, the first or second guide pair 37 or 38 in contact with the roller 39 is selectively moved upward and therefore the top ends of the first or second guide pair 37 or 38 are projected upward from the conveying surface of the conveying table 30. In the state shown in FIG. 4, the roller 39 causes the first guide pair 37 to project from the conveying surface, and the second guide pair 38 is in the retracted state.

The conveyance means 13, according to the type of dry analysis chip, conveys said chip to its appropriate destination. The first dry analysis chip 2 with the dropped inspection matter is conveyed to the distributing section 6 and further from the direction change section 6 to the first incubator 7 by the first conveying member 41 of the conveyance means 13. On the other hand, the second dry analysis chip 3 with the dropped inspection matter is conveyed to the distributing section 6 by the first conveying member 41 and is further conveyed from the distributing section 6 to the second incubator 8 by the second conveying member 42. The first conveying member 41 is constructed of a plate slidably disposed in the longitudinal direction of the conveying table 30, and conveys the dry analysis chip 2 or 3 by forward movement of the plate. A groove 30a is formed lengthwise in the central portion of the conveying table 30, and a slider 43 is mounted on the bottom surface of the first conveying member 41 through the groove 30a. To the rear of the chip stand-by section 4, a guide plate 44 and a cover 45 are disposed above the first conveying member 41.

The slider 43 is slidably supported in the longitudinal direction of the conveying table 30 by a guide rod 46 disposed along the conveying table 30, and is fixedly attached to part of a belt 48 extending between pulleys 47, 47 disposed at the front and rear end portions of the conveying table 30. The rear pulley 47 is rotated by a conveyance motor 49, and the first conveying member 41 is moved in the longitudinal direction by movement of the slider 43. The front end of the first conveying member 41 is pushed against the rear end of the dry analysis chip 2 or 3 and conveys the dry analysis chip 2 or 3. The conveyance motor 49 is controlled so that the dry analysis chip 2 or 3 at the lower end of the chip guide 32 is conveyed to the dropping section 5, the analysis chip 2 or 3 with the dropped inspection matter is conveyed to the distributing section 6, and furthermore, the first dry analysis chip 2 is inserted from the distributing section 6 into the first incubator 7 and the dry analysis chip 2 which has been measured is discarded through the central portion of the first incubator 7. In the case where the dry analysis chip 2 or 3 is conveyed from the dropping section 5 to the distributing section 6 and also from the distributing section 6 to the first incubator 7 by the first conveying member 41, the dry analysis chip 2 or 3 is guided with the first guide pair 37 projected (and with the second guide pair 38 retracted).

On the other hand, the second conveying member 42 (see FIG. 1) is disposed on the side of the distributing section 6 remote from the second incubator 8 and is movable in a direction perpendicular to the conveying direction of the first conveying member 41. Although details are not shown, the second conveying member 42 is driven and controlled by the same drive mechanism as the first conveying member 41 so that it advances and pushes the side surface of the second dry analysis chip 3 being stopped in the distributing section 6, conveys the second dry analysis chip 3 toward the second incubator 8, and after measurement, discards the second dry analysis chip 3 behind the second incubator 8. In the case where the second dry analysis chip 3 is conveyed from the distributing section 6 to the second incubator 8 by the second conveying member 42, the second dry analysis chip 3 is guided with the second guide pair 38 projected (and with the first guide pair 37 retracted). Note that since the distance that the second conveying member 42 is moved is shorter than that of the first conveying member 41, the second conveying member 42 may be provided so that it is moved by another drive mechanism.

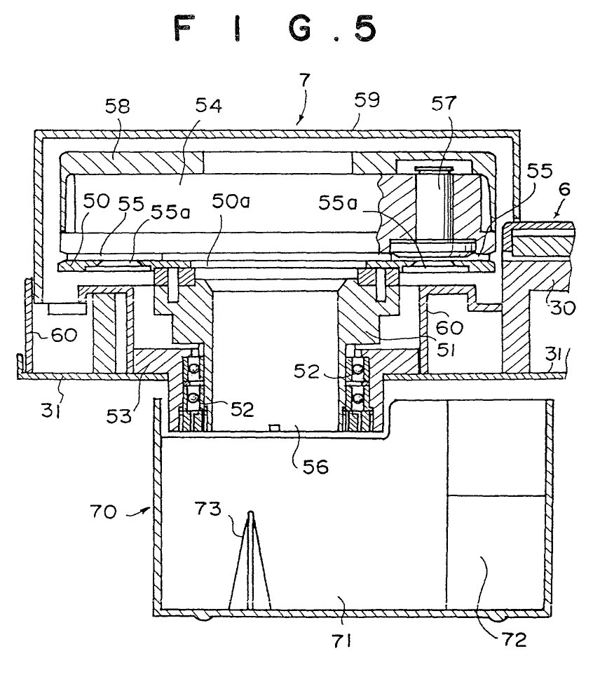

As shown in FIG. 5, the first incubator 7 for making a colorimetric measurement is provided with a disc shaped rotating member 50, and an upper member 54 disposed above the rotating member 50. The rotating member 50 is rotatably supported with respect to a shaft bearing portion 53 through bearings 52 by a rotating cylinder 51. The bottom surface of the upper member 54 is flat, and the top surface of the rotating member 50 has a plurality of recesses (in the case of FIG. 1, six recesses) at predetermined intervals. Chip chambers 55 in the form of a slit are formed between the members 51 and 54. Each chip chamber 55 is provided so that the bottom surface thereof becomes coplanar with the conveying surface of the conveying table 30 of the distributing section 6. The inside bore of the rotating cylinder 51 is formed as a chip discarding bore 56 through which the dry analysis chip 2 which has been measured is discarded. The chip discarding bore 56 is sized so that the dry analysis chip 2 can be passed through it. Also, an opening 50a is formed in the central portion of the rotating member 50 and is communicated with the chip discarding bore 56. The radially inner portion of each chip chamber 55 is communicated with the opening 50a of the rotating member 50 so that if the dry analysis chip 2 in the chip chamber 55 is moved to the opening 50a, it drops into the chip discarding bore 56.

The upper member 54 is equipped with heating means (not shown) so that the first dry analysis chip 2 within the chip chamber 55 is incubated at 37±0.2° C. The upper member 54 is further equipped with a press member 57 which is pressed against the mount of the first dry analysis chip 2 from above to prevent evaporation of the inspection matter dropped on the first dry analysis chip 2. A cover 58 is disposed on the top surface of the upper member 54. The first incubator 7 is provided with an upper cover 59 and a lower cover 60 to intercept light.

Furthermore, a photometric opening 55a is formed in the center of the bottom surface of each chip chamber 55 in which the dry analysis chip 2 is housed. The reflection density of the dry analysis chip 2 is measured through the photometric opening 55a by the photometer head 61 of the measurement means 11 disposed at the position shown in FIG. 1. A white-and-black density reference plate 62 is installed in part of the rotating member 50.

The first incubator 7 is equipped with a timing belt (not shown) wound on the outer periphery of the rotating cylinder 51 which supports the rotating member 50. The timing belt is also wound on the driving pulley (not shown) of a drive motor (not shown). The rotating member 50 is structured to rotate in both directions by rotating the drive motor in forward and backward directions. In the rotational operation of the first incubator 7, the photometer head 61 disposed under the rotating member 50 at a predetermined rotational position is first calibrated by detecting the density of the white-and-black density reference plate 62. Then, the optical density of the color reaction in each of the first dry analysis chips 2 being inserted in the chip chamber 55 is measured in sequence. After the measurement, the rotating member 50 rotates in the backward direction and returns to the reference position for the next measurement. Said rotational operation is controlled to be within a predetermined angular range.

A chip collecting box 70 is disposed under the first incubator 7 to collect the dry analysis chips 2 after measurements have been performed thereon. The chip collecting box 70 has a collecting chamber 71 that is communicated with the chip discarding bore 56 of the rotating cylinder 51. The chip collecting box 70 also has an inclined portion 72, in which the nozzle tip 21 of the dropping means 14 that is exchanged for each inspection matter is dropped. Furthermore, a protrusion 73 is erected in the bottom portion of the collecting chamber 71 to contact with the dry analysis chip 2 being dropped from the chip discarding bore 56 and change the dropping direction of the dry analysis chip 2.

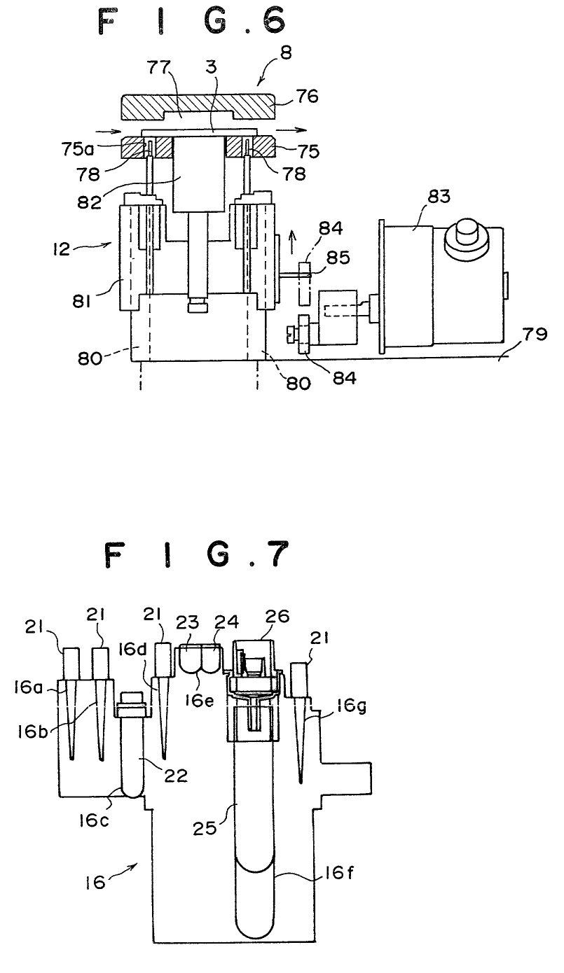

The constructions of the second incubator 8 and the second measurement means 12, for measuring ionic activity, are shown in FIG. 6. The second incubator 8 is equipped with a support member 75 coplanar with the conveying table 30, and an upper member 76 disposed above the support member 75. The support member 75 is used for holding the bottom surface of the second dry analysis chip 3 conveyed from the distributing section 6. A single chip chamber 77 in the form of a slit is formed between the support member 75 and the upper member 76. The rear portion of the chip chamber 77 is open to the outside, and if the second dry analysis chip 3 is moved to the rear portion, it is dropped and discarded. The second incubator 8 is provided with heating means (not shown) so that a portion of the second dry analysis chip 3 where the ionic activity is measured is incubated at 30±0.1° C. within the chip chamber 77. The sides of chip chamber 77 are further equipped with three pairs of measurement openings 75a for measuring ionic activity. The three pairs of measurement openings 75a are provided so that potential measuring probes 78 can make contact with the ion selecting electrode pairs 3c, 3d, 3e of the second dry analysis chip 3.

The second measurement means 12 is equipped with the three potential measuring probes 78 (only one pair is shown) movable in an up-and-down direction. The probe pairs 78 are fixed to a guide member 81, which is guided by a stationary member 80 so that it moves up and down. The stationary member 80 is erected in a base 79. The guide member 81 is provided with a hold member 82 for holding the central portion of the bottom surface of the dry analysis chip 3. As shown in FIG. 6, the bottom surface of the upper member 76 has a recess to minimize the contact between the upper member 76 and the dry analysis chip 3 and to prevent the contact between the upper member 76 and swells of the reference solution and inspection matter in the dry analysis chip 3. The guide member 81 is urged downward by means of a spring (not shown). A drive motor 83 is provided to a side of the guide member 81 and has an output shaft on which a cam member 84 is mounted. This cam member 84 is disposed in opposition to an abutting portion 85 provided on the side portion of the guide member 81. If the drive motor 83 is rotated, the cam member 84 is moved from a position indicated by the solid line to a position indicated by the broken line in FIG. 6. This movement causes the cam member 84 to abut the abutting portion 85, whereby the guide member 81, the probes 78, and the hold member 82 are moved upward. When the cam member 84 is not in contact with the abutting portion 85, the tip ends of the potential measuring probes 78 are in non-contact with the second dry analysis chip 3. On the other hand, if the cam member 84 abuts the abutting portion 85, the tip ends of the potential measuring probes 78 are protruded from the surface of the support member 75 and electrically connected with the ion selecting electrode pairs 3c, 3d, 3c of the second dry analysis chip 3.

The second dry analysis chip 3 with inspection matter in the inspection-matter receiving bore 3a and a reference solution in the reference-solution receiving bore 3b is housed in the chip chamber 77. The three potential measuring probe pairs 78 and the hold member 82 are moved upward so that the second dry analysis chip 3 is held between the hold member 82 and the upper member 76. When this occurs, potential differences are generated between the ion selecting electrode pairs 3c to 3e of the dry analysis chip 3 in accordance with the Cl⁻, K⁺, and Na⁻ differences between the inspection matter and the reference solution. Therefore, if the potential differences generated between the ion selecting electrode pairs 3c to 3e are measured by the three potential measuring probe pairs 78, each ionic activity in the blood plasma can be measured. The ionic activities measured in this manner are displayed on a display panel such as a liquid crystal panel, etc., or recorded on recording paper.

As shown in FIG. 7, the sample housing section 16 is equipped with a first nozzle-tip hold portion 16a for holding a nozzle tip 21 for a reference solution, a second nozzle-tip hold portion 16b for holding a nozzle tip 21 for electrolytic inspection matter, a third nozzle-tip hold portion 16d for holding a nozzle tip 21 for a weak solution, and a fourth nozzle-tip hold portion 16g for holding a nozzle tip 21 for inspection matter. The sample housing section 16 is further equipped with a fifth hold portion 16c for a weak-solution housing tube 22, a sixth hold portion 16e for a reference-solution cup 23 and a mixing cup 24, and a seventh hold portion 16f for a blood-collecting tube 25. The hold portions 16a to 16f are positioned on the swivel orbit of the dropping nozzles 101a, 101b of the dropping arm 96 of the dropping means 14 described later, as shown in FIG. 1. Note that the sample housing section 16 is disposable as a whole. The entire sample housing section 16 is exchangeable with respect to the biochemical analysis apparatus 1a.

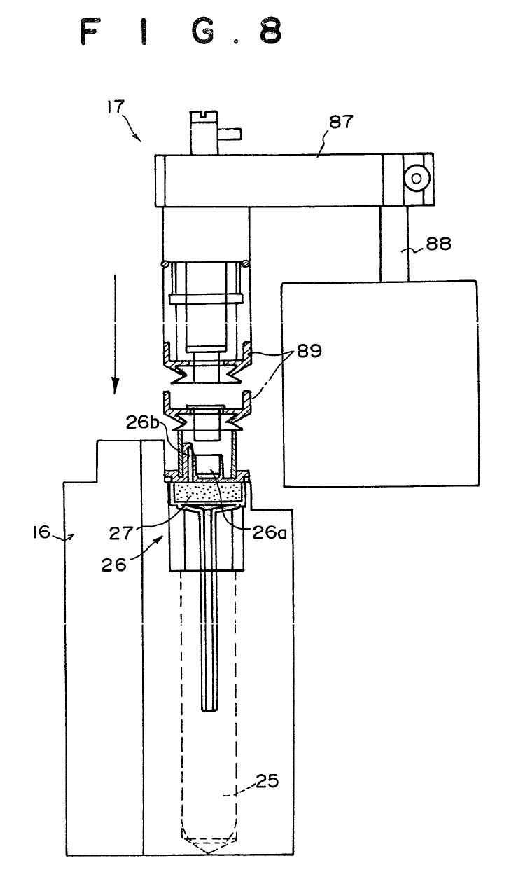

As shown in FIG. 8, the blood plasma filtering unit 17 is inserted into the blood-collecting tube 25 housed in the sample housing section 16, then separates and draws blood plasma from blood through a holder 26, and holds the filtered blood plasma in a cup 26a disposed in the holder 26. The holder 26 has a filter 27 consisting of glass fibers and is mounted in the top opening of the sample housing section 16. A suction arm 87 within which negative pressure is produced has a proximal portion, which is rotatably supported by a supporting shaft 88. The suction arm 87 is provided with a suction disk 89 for holding the holder 26 by suction. The suction disk 89 is connected with a pump (not shown). The suction arm 87 is rotatable through the supporting shaft 88 and a timing belt (not shown) by forward and backward rotations of a drive motor (not shown) and also movable up and down by an elevating mechanism (not shown).

In separating blood plasma from blood, the holder 26 is first set to the blood-collecting tube 25 of the sample housing section 16. Then, the suction arm 87 is rotated so that the suction disk 89 is opposed to the holder 26. Next, the suction arm 87 is moved downward so that it makes direct contact with the holder 26. The whole blood within the blood-collecting tube 25 is drawn up with the drive motor (not shown) and filtered through the filter 27, and the blood plasma is supplied to the cup 26a through a passage 26b. Thereafter, the suction arm 87 is moved upward to the original position, and the filtering process is ended.

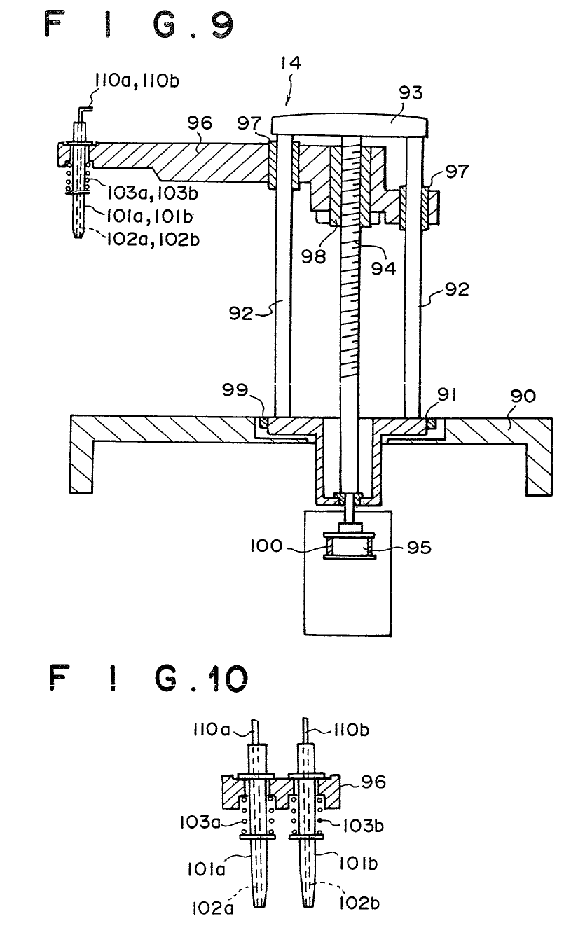

As shown in FIG. 9, the dropping means 14 is equipped with a flange member 91 rotatably attached through bearings (not shown) with respect to a stationary base 90, and guide rods 92 erected in the flange member 91. The upper ends of the guide rods 92 are fixedly attached to a coupling member 93, and the guide rods 92 are disposed parallel to each other in an up-and-down direction. The coupling member 93 is provided with a vertical feed screw 94 at the center portion thereof. The upper end of the feed screw 94 is rotatably supported by the coupling member 93, while the lower end portion is rotatably supported by the center portion of the rotatable flange member 91. Furthermore, the lower tip of the feed screw 94 protrudes from the flange member 91 and a pulley 95 is fixedly attached thereto. The proximal portion of a dropping arm 96 is supported through sleeves 97 by the guide rods 92 so that it is free to move up and down. The feed screw 94 penetrates the dropping arm 96, and the penetrated portion of the arm 96 is provided with a nut member 98 that meshes with the feed screw 94. Thus, the dropping arm 96 is movable up and down according to rotation of the feed screw 94.

As shown in FIG. 10, the outer end portion of the dropping arm 96 is provided with two dropping nozzles 101a, 101b for performing suction and expulsion of inspection matter. The shaft portions of the dropping nozzles 110a, 101b are slidably inserted into the dropping arm 96 and urged downward by springs 103a, 103b. The first dropping nozzle 110a is used for inspection matter and an electrolytic inspection matter, while the second dropping nozzle 101b is used for a weak solution and a reference solution. As described above, the pipette-shaped nozzle tips 21 are detachably attached to the tip ends of the dropping nozzles 101a, 101b. Unused nozzle tips 21 are held in the sample housing section 16, and they are fitted and held on the tip ends of the dropping nozzles 110a, 101b by downward movement of the dropping arm 96. After use, the nozzle tip 21 fitted in the engagement groove of the nozzle-tip removing member 20 (FIG. 1) is separated from the groove by upward movement of the dropping arm 96, and the separated nozzle tip 21 is dropped below the nozzle-tip removing member 20 and is discarded.

The dropping arm 96 is swiveled to a predetermined position through a timing belt 99 extending between the flange member 91 and the driving pulley (not shown) of a drive motor, by rotating the drive motor in forward and backward directions. Also, the dropping arm 96 (i.e., the feed screw 94) is moved to a predetermined height through a timing belt 100 extending between a lower pulley 95 and the driving pulley (not shown) of another drive motor, by rotating the drive motor in forward and backward directions.

To draw and expel inspection matter to and from nozzle tip 21, the central portions of the dropping nozzles 101a, 101b are provided with air passages 102a, 102b open to the tip ends, and the upper ends of the air passages 102a, 102b are connected with air pipes 110a, 110b. The air pipes 110a, 110b are connected with the right end portion (see FIG. 1) of the syringe 105 of syringe means 15. The syringe 105 is a syringe-shaped air pump, and suction and expulsion are performed by operation of the syringe 105. Note that one of the suction passages of the dropping nozzles 101a, 101b is switched to the other with an electromagnetic valve (not shown) provided in the syringe means 15.

The operation of the first embodiment will hereinafter be described in detail. As shown in FIG. 1, the dry analysis chips 2, 3 are put into the chip stand-by section 4, and the disposable sample housing section 16 is prepared. In the sample housing section 16, the nozzle tips 21, the weak-solution housing tube 22, and the blood-collecting tube 25 with blood to be analyzed are held in the hold portions 16a to 16g. Thereafter, the analysis process is started.

Initially, the whole blood within the blood-collecting tube 25 is filtered to obtain the blood plasma component by the blood filtering unit 17. That is, the suction disk 89 of the suction arm 87 is rotated to a position where it faces the holder 26. Then, the suction arm 87 is lowered so that the suction disk 89 is brought into contact with the upper end of the holder 26. If negative pressure is produced within the sucktion arm 87 by driving a pump (not shown), the blood is filtered by the blood filtering unit 17, and the blood plasma is supplied to the cup 26a. Note that a leakage of blood may be detected by checking the pump pressure, or a hematocrit value (volume percent of red cells with respect to whole blood) may be detected. If a predetermined amount of blood plasma is supplied to the cup 26a, the suction arm 87 is moved upward and returned to the original position and the process is ended.

Next, the dry analysis chip 2 or 3 is conveyed from the chip stand-by section 4 to the dropping section 5 by conveying means 13. During the conveyance, the bar code provided in the dry analysis chip 2 or 3 is read by the bar-code reader 35, and the inspection item, etc., of the dry analysis chip 2 or 3 are detected. A different process is performed, depending on the case where the read inspection item indicates ionic activity measurement, the case of a dilution request item, etc.

When the read inspection item indicates coloration measurement, the dropping arm 96 is moved to the sample housing section 16 and the nozzle tip 21 for inspection matter is fitted on the dropping nozzle 101a. The liquid surface of the inspection matter (blood plasma) supplied to the cup 26a is detected to confirm the position of the liquid surface and whether or not a necessary amount of blood plasma has been supplied to the cup 26a. The dropping arm 96 is moved downward and draws the inspection matter from the cup 26a into the nozzle tip 21. Furthermore, the dropping arm 96 with the nozzle tip 21 containing the inspection matter is rotated to the dropping section 5 and drops the inspection matter on the inspection-matter receiving bore 2a of the first dry analysis chip 2.

The first dry analysis chip 2 with the dropped inspection matter is inserted into the first incubator 7. The interior temperature of the first incubator 7 is maintained at 37±0.2° C. for coloration measurement. At this time, it may be detected whether or not the first dry analysis chip 2 has certainly been inserted into the first incubator 7. In the case where dry analysis chips are sequentially processed, they are sequentially conveyed to the dropping section 5 and processed in the same manner. The case where the read inspection item indicates ionic activity measurement, and the case of a weak-solution request item, will be described later.

If the first dry analysis chip 2 is inserted into the first incubator 7, the chip chamber 55 of the first incubator 7 is rotated so that the inserted dry analysis chip 2 is opposed to the photometer head 61. The photometer head 61 measures the optical reflection density of the dry analysis chip 2. After the measurement, the chip chamber 55 is returned to the position where the dry analysis chip 2 was inserted. The measured dry analysis chip 2 is pushed toward the central portion of the first incubator 7 by the first conveying member 41 and is discarded. The result of measurement is output, and the nozzle tip 21 that has been used is removed from the dropping nozzle 101a with the nozzle-tip removing member 20. The removed nozzle tip 21 is dropped and discarded and the process is ended.

In the case where the read inspection item is a dilution request item, for example, in the case where the density of blood plasma is too high to make accurate inspection, the dropping arm 96 is moved to the sample housing section 16 and the nozzle tip 21 for inspection matter is fitted on the dropping nozzle 101a. The liquid surface of the inspection matter (blood plasma) supplied to the cup 26a is detected to confirm the position of the liquid surface and whether or not a necessary amount of blood plasma has been supplied to the cup 26a. The dropping arm 96 is moved downward and draws the inspection matter from the cup 26a into the nozzle tip 21.

Part of the drawn inspection matter is supplied from the nozzle chip 21 into the mixing cup 24. After the division of the inspection matter, the used nozzle chip 21 is removed from the dropping nozzle 101a with the nozzle-tip removing member 20 and is dropped and discarded downward. Next, the dropping arm 96 is moved to the sample housing section 16 and the nozzle tip 21 for a weak solution is fitted on the dropping nozzle 101b. The liquid surface of the weak solution supplied to the weak-solution housing tube 22 is detected to confirm the position of the liquid surface and whether or not a necessary amount of weak solution has been supplied to the weak-solution housing tube 22. The dropping arm 96 is moved downward, and a weak solution is drawn from the weak-solution housing tube 22 and expelled into the weak-solution nozzle tip 21.

The weak solution is expelled from the weak-solution nozzle tip 21 into the mixing cup 24. The weak-solution nozzle tip 21 is inserted within the mixing cup 24, and churning is performed by repeating suction and expulsion. After churning, the diluted inspection matter is drawn by an inspection-matter nozzle tip 21. The dropping arm 96 with the diluted inspection matter is moved to the dropping section 5, and the diluted inspection matter is dropped on the inspection-matter receiving bore 2a of the dry analysis chip 2. In the case where dry analysis chips are sequentially processed, chip conveyance and bar-code reading are performed and the same process is performed. Photometry, discarding of chips, output of results, discarding of nozzle tips are performed in the same manner, and the process is ended.

Next, a description will be given in the case where an inspection item indicates ionic activity measurement. In the case of ionic activity measurement, the second dry analysis chip 3 for ionic activity measurement is conveyed. The dropping arm 96 is moved to the sample housing section 16 and the nozzle tip 21 for an electrolytic inspection matter is fitted on the dropping nozzle 101a. The liquid surface of the inspection matter (blood plasma) supplied to the cup 26a is detected to confirm the position of the liquid surface and whether or not a necessary amount of blood plasma has been supplied to the cup 26a. The dropping arm 96 is moved downward, and inspection matter is drawn from the cup 26a and expelled into the electrolytic-inspection-matter nozzle tip 21.

The electromagnetic value of the syringe means 15 is switched so that the pressure passage is switched to the side of the dropping nozzle 101b. The dropping arm 96 is moved to the sample housing section 16 and the reference-solution nozzle tip 21 is fitted on the dropping nozzle 101b. After the liquid surface of the reference solution supplied to the reference-solution cup 23 has been detected, the dropping arm 96 is lowered and the reference solution is drawn from the reference-solution cup 23 and expelled into the reference-solution nozzle tip 21.

Next, the pressure passage is switched to the side of the dropping nozzle 101a by the electromagnetic valve of the syringe means 15, and the inspection matter that had been drawn into the electrolytic-inspection-matter nozzle tip 21 is dropped on the inspection-matter supply bore 3a of the second dry analysis chip 3. Furthermore, the pressure passage is switched to the side of the dropping nozzle 101b by the electromagnetic valve of the syringe means 15, and the reference solution that had been drawn into the reference-solution nozzle tip 21 is dropped on the reference-solution supply bore 3b of the dry analysis chip 3.

The second dry analysis chip 3 with the inspection matter and the reference solution is moved from the dropping section 5 to the distributing section 6 by the first conveying member 41. Thereafter, the horizontal shaft portion of the roller 39 is rotated through 90° and the first guide pair 37 is retracted from the conveying surface of the conveying table 30 and the second guide pair 38 is projected from the conveying surface. Subsequently, the second conveying member 42 is moved forward and the second dry analysis chip 3 is inserted into the chip chamber 77 of the second incubator 8. The interior temperature of the chip chamber 77 of the second incubator 8 is maintained at 30±1° C. At this time, it may be detected whether or not the second dry analysis chip 3 has certainly been inserted into the chip chamber 77 of the second incubator 8. If the second dry analysis chip 3 is inserted into the second incubator 8, a measurement of ionic activity is made by the second measurement means 12. After the measurement, the measured dry analysis chip 3 is pushed out to the outside by the second conveying member 42 and is discarded from the second incubator 8. The result of measurement is output, and the reference-solution nozzle tip 21 and the electrolytic-inspection-matter nozzle tip 21 that have been used are removed from the dropping nozzle 101a with the nozzle-tip removing member 20. The removed nozzle tips 21 are dropped and discarded and the process is ended.

Thus, in the first embodiment, the bar code of the dry analysis chip 2 or 3 is read and the type is identified. According to the type, dropping of inspection matter is performed. Also, the first dry analysis chip 2 is inserted into the first incubator 7 by the first conveying member 41 of the conveyance means 13, while the second dry analysis chip 3 is inserted from the distributing section 6 into the second incubator 8 by the second conveying member 42. The first and second dry analysis chips 2, 3 are incubated at their respective incubation temperatures, and the calorimetric measurement and potential difference measurement are made by the first measurement means 11 and the second measurement means 12 to detect the substance density and ionic activity. When the conveying direction of the second dry analysis chip 3 is changed by the distributing section 6, the first guide pair 37 is switched to the second guide pair 38. Thus, the dry analysis chips 2, 3 can be reliably conveyed. In addition, the substance density measurement and the ionic activity measurement can be simultaneously made by the first measurement means 11 and the second measurement means 12, so the dry analysis chips 2, 3 can be efficiently processed with compact construction.

The number of dry analysis chips 2, 3 that are housed in the first and second incubators 7, 8 in the first embodiment is arbitrary. However, it is preferable from the actual ratio of measurement to hold six analysis chips in the first incubator 7 and one analysis chip in the second incubator 8.

While the conveyance of the second dry analysis chip 3 to the second incubator 8 is perpendicular to the conveying direction of the first dry analysis chip 2, the present invention is not limited to this direction. The second dry analysis chip 3 can be conveyed at any predetermined angle.

In the first embodiment, the first and second dry analysis chips 2, 3 are incubated at different temperatures by the first and second incubators 7, 8. However, they may be incubated at the same temperature by the first and second incubators 7, 8.

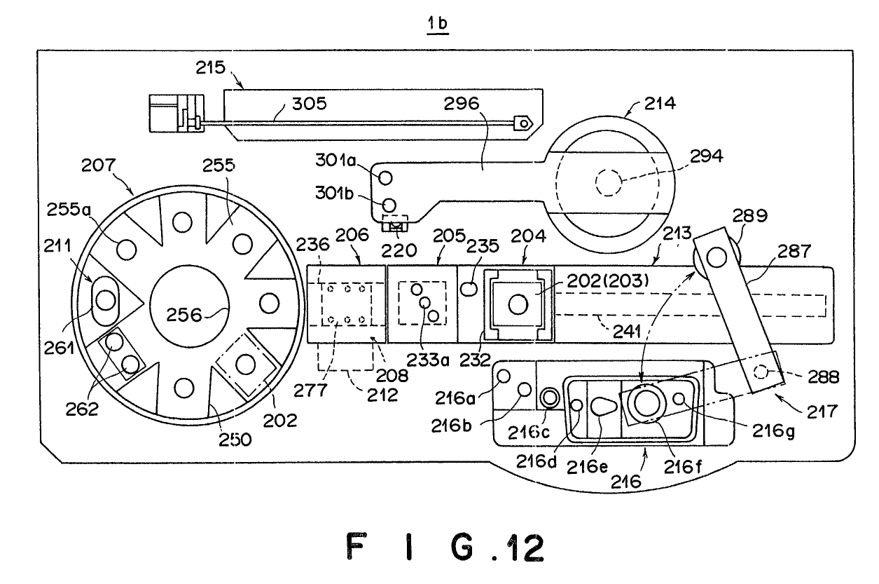

FIG. 12 illustrates a biochemical analysis apparatus 1b constructed according to a second embodiment of the present invention. The biochemical analysis apparatus 1b is equipped with a chip stand-by section (chip housing section) 204 for housing a first dry analysis chip (colorimetric-type dry analysis chip) 202 and a second dry analysis chip (electrolytic-type dry analysis chip) 203 in a mixed state; a dropping section 205, following the chip stand-by section 204, for dropping inspection matter (which can be whole blood, serum, urine, blood plasma, etc., but, in the second embodiment, only blood plasma will be explained) on the dry analysis chips 202, 203; and a distributing section 206, following the dropping section 205, for separating the first dry analysis chip 202 and the second dry analysis chip 203. The biochemical analysis apparatus 1b is also equipped with a first incubator 207, disposed in the distributing section 206, for incubating the first dry analysis chip 202 for a predetermined time; a second incubator 208, disposed near the side surface of the distributing section 206, for incubating the second dry analysis chip 203 for a predetermined time; first measurement means 211 for measuring the first dry analysis chip 202 installed in the first incubator 207; and second measurement means 212 for measuring the second dry analysis chip 203 installed in the second incubator 208.

The biochemical analysis apparatus 1b is further equipped with conveyance means 213 for conveying the first dry analysis chip 202 and the second dry analysis chip 203 to the first incubator 207 and the second incubator 208.

The conveyance means 213 conveys the first and second analysis chips 202, 203 in sequence from the chip stand-by section 204 to the dropping section 205 by forward movement of an insertion member 241. In the dropping section 205, inspection matter is dropped on the first dry analysis chip 202 by dropping means (sampler) 214, and inspection matter and a reference solution are dropped on the second dry analysis chip 203 by the dropping means 214. Thereafter, the first dry analysis chip 202 with the dropped inspection matter is linearly inserted into the first incubator 207 through the distributing section 206 by the insertion member 241. The second dry analysis chip 203 is inserted into the chip chamber 277 of the second incubator 208 of the distributing section 206 by the insertion member 241.

The coloration (optical reflection density) of the first dry analysis chip 202 incubated by the first incubator 207 is measured with the first measurement means 211, and a potential difference in the second dry analysis chip 203 incubated by the second incubator 208 is measured with the second measurement means 212. After the measurements, the first dry analysis chip 202 is further conveyed by the insertion member 241 and is dropped and discarded through the center portion of the first incubator 207. The second dry analysis chip 203 is likewise conveyed to the center portion of the first incubator 207 by the insertion member 241 and is dropped and discarded through the central portion.

The distributing section 206 has a passage 236 for conveying the first dry analysis chip 202 to the first incubator 207, and also has a chip chamber 277 for the second incubator 208, disposed under the passage 236. The distributing section 206 is movable up and down, depending on analysis chip type (see FIG. 13). When the second dry analysis chip 203 is conveyed from the dropping section 205 by the conveyance means 213, the distributing section 206 is moved upward so that the second dry analysis chip 203 is received in the chip chamber 277 of the second incubator 207. When the first dry analysis chip 202 is conveyed from the dropping section 205 by the conveyance means 213, the first dry analysis chip 202 is inserted into the first incubator 207 through the passage 237, or through the unoccupied chip chamber 277.

In the case where the second dry analysis chip 203 has already been inserted in the chip chamber 277 when the first dry analysis chip 202 is being conveyed, the first dry analysis chip 202 is conveyed through the passage 236, because the distributing section 206 has been lowered. However, when the chip chamber 277 has not been occupied, the first dry analysis chip 202 can be conveyed through the chip chamber 277. In consideration of efficiency, the chip chamber 203 is normally operated at the height of the conveying path so that the first dry analysis chip 202 can be passed through the chip chamber 203. And when the second dry analysis chip 203 is conveyed, the distributing section 206 is lowered. During the time that the second dry analysis chip 203 in the second incubator 208 is being measured by the second measurement means 212, the next dry analysis chip 202 is inserted into the first incubator 207 through the lowered passage 236.

The dropping means 214 fits a nozzle tip 221 (described later) onto the tip end of a dropping nozzle 301a or 301b, then draws inspection matter (blood plasma), a reference solution, etc., from a sample housing section 216 into the nozzle tip 221, and drops a predetermined quantity of inspection matter on the dry analysis chip 202 or 203. To drop inspection matter, the dropping means 214 is provided with syringe means 215 which draws and expels the inspection matter by the nozzle tip 221. After it is used, the nozzle tip 221 is removed by a nozzle-tip removing section 220 and is dropped and discarded downward. Also, a blood filtering unit 217 is disposed near the sample housing section 216 to separate blood plasma from blood.

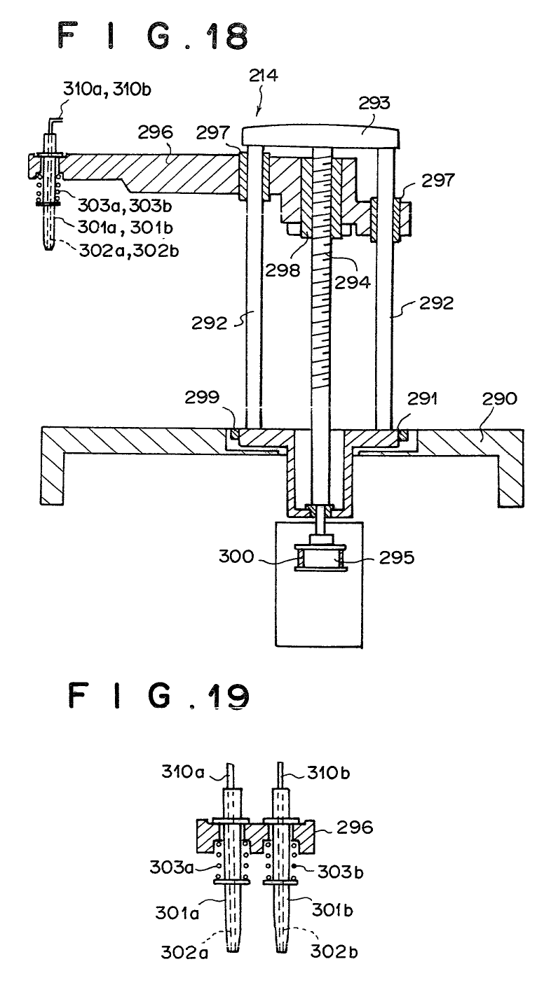

Now, the construction of the first dry analysis chip 202 of the calorimetric type used for measuring the coloration of blood plasma, and the construction of the second dry analysis chip 203 of the electrolytic type used for measuring the ionic activity of blood plasma, will be described with reference to FIG. 20. The first dry analysis chip 202 on the left side is constructed of a rectangular mount within which a layer of reagent is disposed. The mount has an inspection-matter receiving bore 202a to which inspection matter is dropped. On the other hand, the second dry analysis chip 203 on the right side is approximately the same in shape as the first dry analysis chip 202, and has an inspection-matter receiving bore 203a to which inspection matter is dropped, and a reference-solution receiving bore 203b to which a reference solution with known ionic activity is dropped. The second dry analysis chip 203 also has three pairs of ion selecting electrodes 203c, 203d, 203e, which are connected electrically with the potential measuring probes of the second measurement means 212 to measure ionic activity. The ion selecting electrode pairs 203c, 203d, 203e have Cl⁻-, K⁺-, and Na⁺-selecting layers, respectively. Also, the bottom surfaces of the first and second dry analysis chips 202, 203 are each provided with a bar code representing information that identifies an inspection item, etc.

Next, the construction of each section of the biochemical analysis apparatus 1b will be described. Initially, the chip stand-by section 204, the dropping section 205, the distributing section 206, and the conveyance means 213 will be described with reference to FIG. 13. The conveyance means 213 is equipped with a conveying table 230 linearly extending toward the center of the first incubator 207. The conveying table 230 is installed on a flat base 231. The chip stand-by section 204 is disposed in approximately the central portion of the conveying table 230, and the dropping section 205 and the distributing section 206 are disposed on the side of the conveying table 230 closer to the first incubator 207 than the central portion of the conveying table 230.

The chip stand-by section 204 is provided with a chip guide 232. In the chip guide 232, a plurality of unused first and second dry analysis chips 202, 203 are stacked and held in a mixed state. Also, the lowermost analysis chip 202 or 203 in the chip guide 232 is coplanar with the conveying surface of the conveying table 230. The front wall of the chip guide 232 has a front slit through which only a single analysis chip 202 or 203 is passed, while the rear wall has a rear slit into which the insertion member 241 is inserted. Note that a cartridge with a plurality of dry analysis chips 202, 203 stacked together may be provided in the chip guide 232.

The dropping section 205 is provided with a chip press 233 that has three dropping bores 233a. The chip press 233 is housed within a cover 234 fixedly attached above the conveying table 230. A bar-code reader 235 is interposed between the chip stand-by section 204 and the dropping section 205 in order to read the bar code of the dry analysis chip 202 or 203. The bar-code reader 235 is provided for specifying an inspection item, etc., also controlling the dropping of inspection matter and a reference solution, conveyance, and measurement, and detecting the conveying direction, etc., of the dry analysis chip 202 or 203 (e.g., the forward direction, rearward direction, top surface, bottom surface, etc.).

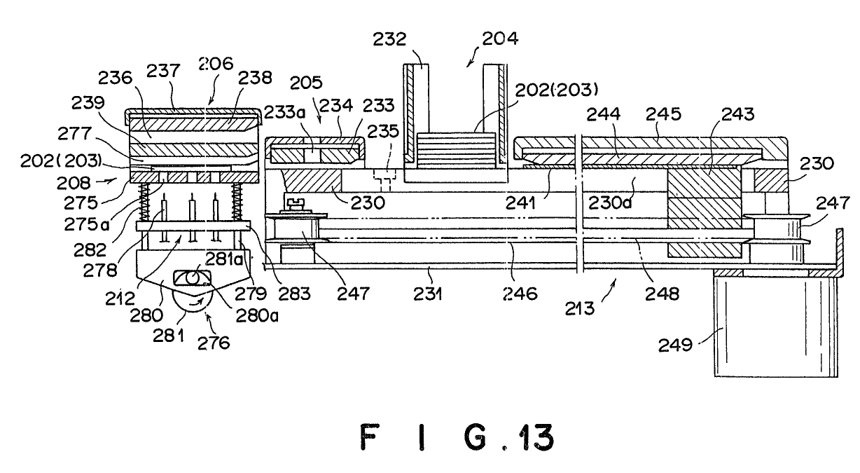

The operating state of the distributing section 206 is shown in FIG. 14. As previously described, the distributing section 206 is provided with the passage 236 above and the second incubator 277 below, movable up and down. A chip press 238 is provided within an upper cover 237. An intervening member 239 is provided so that the passage 236 is formed between it and the chip press 238. A support member 275 is disposed under the intervening member 239 so that the chip chamber 277 is formed therebetween. The chip press 238, the intervening member 239, and the support member 275 are formed integrally with one another and are held so that they are movable up and down as a whole. They are connected with an elevating mechanism 276 and movable up and down.

The elevating mechanism 276 is equipped with a lower elevating member 280 connected with the support member 275 through rods 279. The elevating member 280 has an elongated bore 280a into which the eccentric protrusion 281a of a cam member 281 is inserted. The cam member 281 is rotated by a drive motor (not shown), whereby the elevating member 281 is moved down from an upper position of FIG. 13 to a lower position of FIG. 14. The support member 275 is urged upward by means of a spring 282. The second incubator 208 and the second measurement means 212 will be described later.

The first dry analysis chip 202 with the dropped inspection matter is conveyed to the distributing section 206 and further from the direction change section 206 to the first incubator 207 by the insertion member 241 of the conveyance means 213. On the other hand, the second dry analysis chip 203 with the dropped inspection matter is conveyed to the distributing section 206 by the insertion member 241 and is stopped. The insertion member 241 is constructed of a plate slidably disposed in the longitudinal direction of the conveying table 230, and conveys the dry analysis chip 202 or 203 by forward movement of the plate. A groove 230a is formed lengthwise in the central portion of the conveying table 230, and a slider 243 is mounted on the bottom surface of the insertion member 241 through the groove 230a. Near the chip stand-by section 204, a guide plate 244 and a cover 245 are disposed above the insertion member 241.

The slider 243 is slidably supported in the longitudinal direction of the conveying table 230 by a guide rod 246 disposed along the conveying table 230, and is fixedly attached to part of a belt 248 extending between pulleys 427, 247 disposed at the front and rear end portions of the conveying table 230. The rear pulley 247 is rotated by a conveyance motor 249, and the insertion member 241 is moved in the longitudinal direction by movement of the slider 243. The front end of the insertion member 241 is pushed against the rear end of the dry analysis chip 202 or 203 and conveys the dry analysis chip 202 or 203. The conveyance motor 249 is controlled so that the dry analysis chip 202 or 203 at the lower end of the chip guide 232 is conveyed to the dropping section 205, also the analysis chip 202 or 203 with the dropped inspection matter is conveyed to the distributing section 206, and furthermore, the first and second dry analysis chips 202, 203 measured are discarded through the central portion of the first incubator 207.

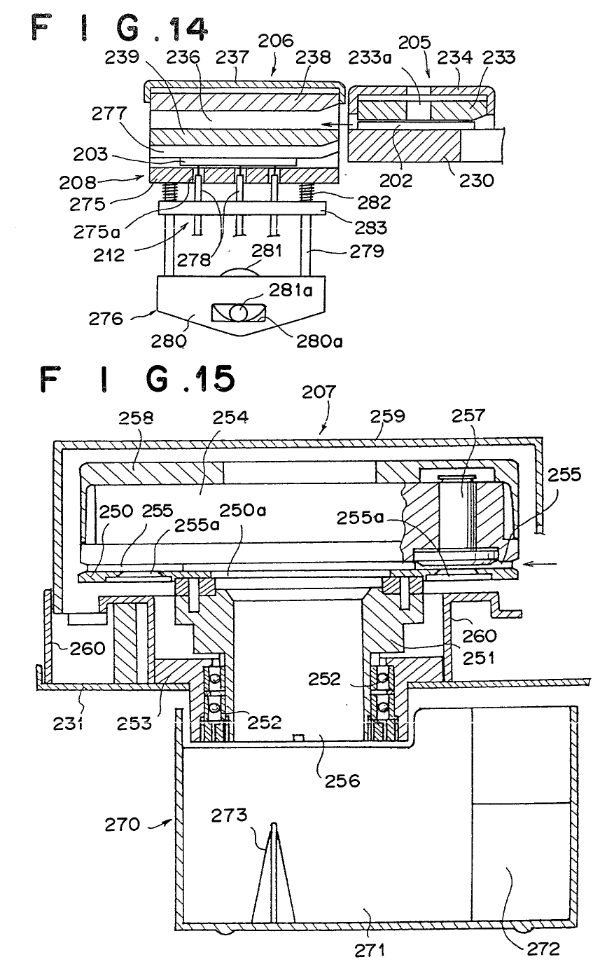

As shown in FIG. 15, the first incubator 207 for making a calorimetric measurement is provided with a rotating member 250, and an upper member 254 disposed above the rotating member 250. The rotating member 250 is rotatably supported with respect to a shaft bearing portion 253 through bearings 252 by a rotating cylinder 251. The bottom surface of the upper member 254 is flat, and the top surface of the rotating member 250 has a plurality of recesses (in the case of FIG. 12, six recesses) at predetermined intervals. Chip chambers 255 in the form of a slit are formed between the members 251 and 254. Each chip chamber 255 is provided so that the bottom surface thereof becomes coplanar with the conveying surface of the conveying table 230 of the distributing section 206. The inside bore of the rotating cylinder 251 is formed as a chip discarding bore 256 through which the dry analysis chip 202 which has been measured is discarded. The chip discarding bore 256 is sized so that the dry analysis chips 202, 203 can be passed through it. Also, an opening 250a is formed in the central portion of the rotating member 250 and is communicated with the chip discarding bore 256. The radially inner portion of each chip chamber 255 is communicated with the opening 250a of the rotating member 250 so that if the dry analysis chip 202 in the chip chamber 255, as it is, is moved to the opening 250a, it is dropped into the chip discarding bore 256.

The upper member 254 is equipped with heating means (not shown) so that the first dry analysis chip 202 within the chip chamber 255 is incubated at 37±0.2° C. The upper member 254 is further equipped with a press member 257 which is pressed against the mount of the first dry analysis chip 202 to prevent evaporation of the inspection matter dropped on the first dry analysis chip 202. A cover 258 is disposed on the top surface of the upper member 254. The first incubator 207 is provided with an upper cover 259 and a lower cover 260 to intercept light.

Furthermore, a photometric opening 255a is formed in the center of the bottom surface of each chip chamber 255 in which the dry analysis chip 202 is housed. The reflection density of the dry analysis chip 202 is measured through the photometric opening 255a by the photometer head 261 of the measurement means 211 disposed at the position shown in FIG. 12. A white-and-black density reference plate 262 is installed in part of the rotating member 250.

The first incubator 207 is equipped with a timing belt (not shown) wound on the outer periphery of the rotating cylinder 251 which supports the rotating member 250. The timing belt is also wound on the driving pulley (not shown) of a drive motor (not shown). The rotating member 250 is rotated in both directions by rotating the drive motor in forward and backward directions. In the rotational operation of the first incubator 207, the photometer head 261 disposed under the rotating member 250 at a predetermined rotational position is first calibrated by detecting the density of the white-and-black density reference plate 262. Then, the optical density of the color reaction in each of the first dry analysis chips 202 which have been inserted in the chip chamber 255 is measured in sequence. After the measurement, the rotating member 250 rotates in the backward direction and returns to the reference position for the next measurement.

A chip collecting box 270 is disposed under the first incubator 207 to collect the dry analysis chips 202 after measurements have been performed thereon. The chip collecting box 270 has a collecting chamber 271 that is communicated with the chip discarding bore 256 of the rotating cylinder 251. The chip collecting box 270 also has an inclined portion 272, in which the nozzle tip 221 of the dropping means 214 that is exchanged for each inspection matter is dropped. Furthermore, a protrusion 273 is erected in the bottom portion of the collecting chamber 271 to contact with the dry analysis chip 202 being dropped from the chip discarding bore 256 and change the dropping direction of the dry analysis chip 202.

As shown in FIGS. 13 and 14, the second incubator 208 for measuring ionic activity is disposed in the distributing section 206. The single chip chamber 277 in the form of a slit is formed between the support member 275 and the intervening member 239. The rear portion of the chip chamber 277 is open to the outside, and if the second dry analysis chip 203, as it is, is moved to the rear portion, it is inserted into the chip chamber 255 of the first incubator 207. If it is further conveyed, it is dropped and discarded through the central portion of the first incubator 207. The second incubator 208 is provided with heating means (not shown) so that a portion of the second dry analysis chip 203 where the ionic activity is measured is incubated at 30±0.1° C. within the chip chamber 277. The sides of chip chamber 277 are further equipped with three pairs of measurement openings 275a for measuring ionic activity. The three pairs of measurement openings 275a are provided so that three potential measuring probe pairs 278 can make contact with the ion selecting electrode pairs 203c, 203d, 203e of the second dry analysis chip 203.

The second measurement means 212 is equipped with three pairs of potential measuring probes 278 (only one side is shown). The potential measuring probes 278 are erected in a stationary frame 283. In the state of FIG. 14 in which the support member 275 has been lowered by the elevating mechanism 276, the probes 278 are in contact with the dry analysis chip 203.

That is, if the cam member 281 is rotated by the drive motor, the elevating member 280 is lowered and the support member 275 is moved downward. In the case of FIG. 13 in which the support member 275 is held in the raised position, the tip ends of the potential measuring probes 278 are in non-contact with the second dry analysis chip 203. However, if the support member 275 is lowered, the tip ends of the potential measuring probes 278 are projected through the openings 275a of the support member 275 and are contacted electrically with the ion selecting electrode pairs 203c to 203e of the dry analysis chip 203.

The second dry analysis chip 203 with inspection matter in the inspection-matter receiving bore 203a and a reference solution in the reference-solution receiving bore 203b is housed in the chip chamber 277 at the raised position. Thereafter, if the second dry analysis chip 203 is lowered, it is contacted with the three potential measuring probes 278 positioned downward. When this occurs, potential differences are generated between the ion selecting electrode pairs 203c to 203e of the dry analysis chip 20 in accordance with the Cl⁻, K⁺, and Na⁻ differences between the inspection matter and the reference solution. Therefore, if the potential differences generated between the ion selecting electrode pairs 203c to 203e are measured by the three potential measuring probe pairs 278, each ionic activity in the blood plasma can be measured. The ionic activities measured in this manner are displayed on a display panel such as a liquid crystal panel, etc., or recorded on recording paper.

As shown in FIG. 16, the sample housing section 216 is equipped with a first nozzle-tip hold portion 216a for holding a nozzle tip 221 for a reference solution, a second nozzle-tip hold portion 216b for holding a nozzle tip 221 for electrolytic inspection matter, a third nozzle-tip hold portion 216d for holding a nozzle tip 221 for a weak solution, and a fourth nozzle-tip hold portion 216g for holding a nozzle tip 221 for inspection matter. The sample housing section 216 is further equipped with a fifth hold portion 216c for a weak-solution housing tube 222, a sixth hold portion 216e for a reference-solution cup 223 and a mixing cup 224, and a seventh hold portion 216f for a blood-collecting tube 225. The hold portions 216a to 216f are positioned on the swivel orbit of the dropping nozzles 201a, 201b of the dropping arm 296 of the dropping means 214 described later, as shown in FIG. 12. Note that the sample housing section 216 is disposable as a whole. The entire sample housing section 126 is exchangeable with respect to the biochemical analysis apparatus 1b.

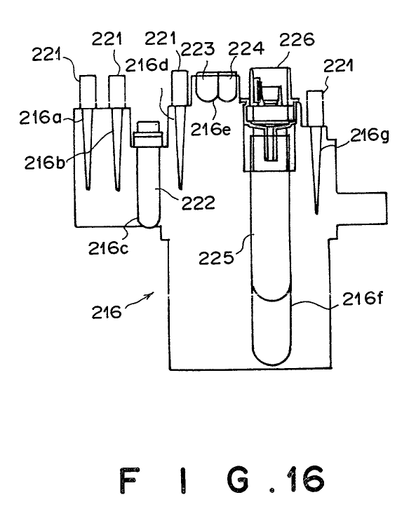

As shown in FIG. 17, the blood plasma filtering unit 217 is inserted into the blood-collecting tube 225 housed in the sample housing section 216, then separates and sucks blood plasma from blood through a holder 226, and holds the filtered blood plasma in a cup 226a disposed in the holder 226. The holder 226 has a filter 227 consisting of glass fibers and is mounted in the top opening of the sample housing section 216. A sucking arm 287 within which negative pressure is produced has a proximal portion, which is rotatably supported by a supporting shaft 288. The sucking arm 287 is provided with a suction disk 289 for attacking the holder 226 by suction. The suction disk 289 is connected with a pump (not shown). The suction arm 287 is rotatable through the supporting shaft 288 and a timing belt (not shown) by forward and backward rotations of a drive motor (not shown) and also movable up and down by an elevating mechanism (not shown).

In separating blood plasma from blood, the holder 226 is first set to the blood-collecting tube 225 of the sample housing section 216. Then, the suction arm 287 is rotated so that the suction disk 289 is opposed to the holder 226. Next, the suction arm 287 is moved downward so that it makes direct contact with the holder 226. The whole blood within the blood-collecting tube 225 is drawn with the drive motor (not shown) and filtered through the filter 227, and the blood plasma is supplied to the cup 226a through a passage 226b. Thereafter, the suction arm 287 is moved upward to its original position, and the filtering process is ended.

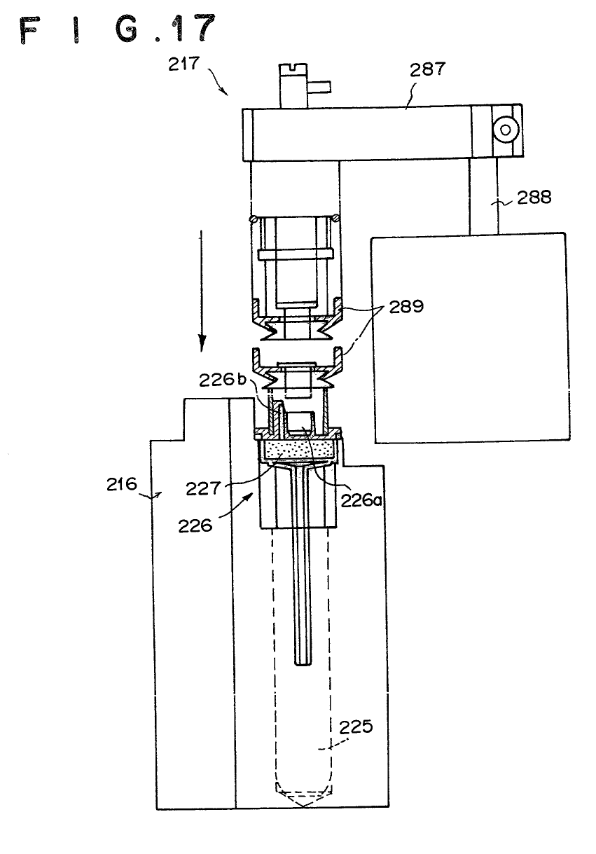

As shown in FIG. 18, the dropping means 214 is equipped with a flange member 291 rotatably attached through bearings (not shown) with respect to a stationary base 290, and guide rods 292 erected in the flange member 291. The upper ends of the guide rods 292 are fixedly attached to a coupling member 293, and the guide rods 292 are disposed parallel to each other in an up-and-down direction. The coupling member 293 is provided with a vertical feed screw 294 at the center portion thereof. The upper end of the feed screw 294 is rotatably supported by the coupling member 293, while the lower end portion is rotatably supported by the center portion of the rotatable flange member 291. Furthermore, the lower end of the feed screw 294 protrudes from the flange member 291 and is provided with a pulley 295. The proximal portion of a dropping arm 296 is supported through sleeves 297 by the guide rods 292 so that it is free to move up and down. The feed screw 294 penetrates the dropping arm 296, and the penetrated portion of the arm 296 is provided with a nut member 298 that meshes with the feed screw 294. Thus, the dropping arm 296 is movable up and down according to rotation of the feed screw 294.

As shown in FIG. 19, the outer end portion of the dropping arm 296 is provided with two dropping nozzles 301a, 301b for performing suction and expulsion of inspection matter. The shaft portions of the dropping nozzles 301a, 301b are slidably inserted into the dropping arm 296 and urged downward by springs 303a, 303b. The first dropping nozzle 301a is used for inspection matter and an electrolytic inspection matter, while the second dropping nozzle 301b is used for a weak solution and a reference solution. As described above, the pipette-shaped nozzle tips 221 are detachably attached to the tip ends of the dropping nozzles 301a, 301b. Unused nozzle tips 221 are held in the sample housing section 216, and they are fitted and held on the tip ends of the dropping nozzles 301a, 301b by downward movement of the dropping arm 296. After use, the nozzle tip 221 fitted in the engagement groove of the nozzle-tip removing section 220 (FIG. 1) is separated from the groove by upward movement of the dropping arm 296, and the separated nozzle tip 221 is dropped below the nozzle-tip removing section 220 and is discarded.

The dropping arm 296 is swiveled to a predetermined position through a timing belt 299 extending between the flange member 291 and the driving pulley of a drive motor, by rotating the drive motor forward and backward directions. Also, the dropping arm 296 (i.e., the feed screw 294) is moved to a predetermined height through a timing belt 300 extending between a lower pulley 295 and the driving pulley of another drive motor, by rotating the drive motor forward and backward directions.

To draw and expel inspection matter, the central portions of the dropping nozzles 301a, 301b are provided with air passages 302a, 302b open to the tip ends, and the upper ends of the air passages 302a, 302b are connected with air pipes 110a, 310b. The air pipes 310a, 310b are connected with the right end portion (see FIG. 12) of the syringe 305 of syringe means 215. The syringe 305 is a syringe-shaped air pump, and suction and expulsion are performed by operation of the syringe 305. Note that one of the suction passages of the dropping nozzles 301a, 301b is switched to the other with an electromagnetic valve (not shown) provided in the syringe means 215.

The operation of the second embodiment will hereinafter be described in detail. As shown in FIG. 12, the dry analysis chips 202, 203 are put into the chip stand-by section 204, and the sample housing section 216 that is a disposable type is prepared. In the sample housing section 216, the nozzle chips 221, the weak-solution housing tube 222, and the blood-collecting tube 225 with blood to be analyzed are held in the hold portions 216a to 216g. Thereafter, the analysis process is started.

Initially, the whole blood within the blood-collecting tube 225 is filtered to obtain the blood plasma component by the blood filtering unit 217. That is, the suction disk 289 of the suction arm 287 is rotated to a position where it faces the holder 226. Then, the suction arm 287 is lowered so that the suction disk 289 is brought into contact with the upper end of the holder 226. If negative pressure is produced within the suction arm 287 by driving a pump (not shown), the blood is filtered by the blood filtering unit 217, and the blood plasma is supplied to the cup 226a. Note that a leakage of blood may be detected by checking the pump pressure, or a hematocrit value(volume percent of red cell with respect to whole blood) may be detected. If a predetermined amount of blood plasma is supplied to the cup 226a, the suction arm 287 is moved upward and returned to its original position and the process is ended.

Next, the dry analysis chip 202 or 203 is conveyed from the chip stand-by section 204 to the dropping section 205. During the conveyance, the bar code provided in the dry analysis chip 202 or 203 is read by the bar-code reader 235, and the inspection item, etc., of the dry analysis chip 202 or 203 are detected. A different process is performed, depending on the case where the read inspection item indicates ionic activity measurement, the case of a dilution request item, etc.

When the read inspection item indicates coloration measurement, the dropping arm 296 is moved to the sample housing section 216 and the nozzle tip 221 for inspection matter is fitted on the dropping nozzle 301a. The liquid surface of the inspection matter (blood plasma) supplied to the cup 226a is detected to confirm the position of the liquid surface and whether or not a necessary amount of blood plasma has been supplied to the cup 226a. The dropping arm 296 is moved downward and draws the inspection matter from the cup 226a into the nozzle tip 221. Furthermore, the dropping arm 296 with the nozzle tip 221 containing the inspection matter is rotated to the dropping section 205 and drops the inspection matter on the inspection-matter receiving bore 202a of the first dry analysis chip 202.

The first dry analysis chip 202 with the dropped inspection matter is inserted into the first incubator 207 in the distributing section 206, or into the first incubator 207 through the passage 236. The interior temperature of the first incubator 207 is maintained at 37±0.2° C. for coloration measurement. At this time, it may be detected whether or not the first dry analysis chip 202 has certainly been inserted into the first incubator 207. In the case where dry analysis chips are sequentially processed, they are sequentially conveyed to the dropping section 205 and processed in the same manner. The case where the read inspection item indicates ionic activity measurement, and the case of a weak-solution request item, will be described later.

If the first dry analysis chip 202 is inserted into the first incubator 207, the chip chamber 255 of the first incubator 207 is rotated so that the inserted dry analysis chip 202 is opposed to the photometer head 261. The photometer head 261 measures the optical reflection density of the dry analysis chip 202. After the measurement, the chip chamber 255 is returned to the position where the dry analysis chip 202 was inserted. The measured dry analysis chip 202 is pushed toward the central portion of the first incubator 207 by the insertion member 241 and is discarded. The result of measurement is output, and the nozzle tip 221 that has been used is removed from the dropping nozzle 301a with the nozzle-tip removing section 220. The removed nozzle tip 221 is dropped and discarded and the process is ended.

In the case where the read inspection item is a dilution request item, for example, in the case where the density of blood plasma is too high to make accurate inspection, the dropping arm 296 is moved to the sample housing section 216 and the nozzle tip 221 for inspection matter is fitted on the dropping nozzle 301a. The liquid surface of the inspection matter (blood plasma) supplied to the cup 226a is detected to confirm the position of the liquid surface and whether or not a necessary amount of blood plasma has been supplied to the cup 226a. The dropping arm 296 is moved downward and draws the inspection matter from the cup 226a into the nozzle tip 221.

Part of the drawn inspection matter is supplied from the nozzle chip 221 into the mixing cup 224. After the division of the inspection matter, the nozzle chip 221 used is removed from the dropping nozzle 301a with the nozzle-tip removing section 220 and is dropped and discarded downward. Next, the dropping arm 296 is moved to the sample housing section 216 and the nozzle tip 221 for a weak solution is fitted on the dropping nozzle 301b. The liquid surface of the weak solution supplied to the weak-solution housing tube 222 is detected to confirm the position of the liquid surface and whether or not a necessary amount of weak solution has been supplied to the weak-solution housing tube 222. The dropping arm 296 is moved downward, and a weak solution is drawn from the weak-solution housing tube 222 and expelled into the weak-solution nozzle tip 221.

The weak solution is expelled from the weak-solution nozzle tip 221 into the mixing cup 224. The weak-solution nozzle tip 221 is inserted within the mixing cup 224, and churning is performed by repeating suction and expulsion. After churning, the diluted inspection matter is drawn by an inspection-matter nozzle tip 221. The dropping arm 296 with the diluted inspection matter is moved to the dropping section 205, and the diluted inspection matter is dropped on the inspection-matter receiving bore 202a of the dry analysis chip 202. In the case where dry analysis chips are sequentially processed, chip conveyance and bar-code reading are performed and the same process is performed. Photometry, discarding of chips, output of results, discarding of nozzle tips are performed in the same manner, and the process is ended.

Next, a description will be given in the case where an inspection item indicates ionic activity measurement. In the case of ionic activity measurement, the second dry analysis chip 203 for ionic activity measurement is conveyed. The dropping arm 296 is moved to the sample housing section 216 and the nozzle tip 221 for an electrolytic inspection matter is fitted on the dropping nozzle 301a. The liquid surface of the inspection matter (blood plasma) supplied to the cup 226a is detected to confirm the position of the liquid surface and whether or not a necessary amount of blood plasma has been supplied to the cup 226a. The dropping arm 296 is moved downward, and inspection matter is drawn from the cup 226a and expelled into the electrolytic-inspection-matter nozzle tip 221.

The electromagnetic value of the syringe means 215 is switched so that the pressure passage is switched to the side of the dropping nozzle 301b. The dropping arm 296 is moved to the sample housing section 216 and the reference-solution nozzle tip 221 is fitted on the dropping nozzle 301b. After the liquid surface of the reference solution supplied to the reference-solution cup 223 has been detected, the dropping arm 296 is lowered and the reference solution is drawn from the reference-solution cup 223 and expelled into the reference-solution nozzle tip 221.

Next, the pressure passage is switched to the side of the dropping nozzle 301a by the electromagnetic valve of the syringe means 215, and the inspection matter which had been drawn into the electrolytic-inspection-matter nozzle tip 221 is dropped on the inspection-matter supply bore 203a of the second dry analysis chip 203. Furthermore, the pressure passage is switched to the side of the dropping nozzle 301b by the electromagnetic valve of the syringe means 215, and the reference solution which had been drawn into the reference-solution nozzle tip 221 is dropped on the reference-solution supply bore 203b of the dry analysis chip 203.

The second dry analysis chip 203 with the inspection matter and the reference solution is moved from the dropping section 205 to the distributing section 206 and inserted into the chip chamber 277 by the insertion member 241. When the second dry analysis chip 203 is conveyed from the dropping section 205, the distributing section 206 is moved upward so that the chip chamber 277 becomes coplanar with the conveying surface of the conveying table 230. The interior temperature of the chip chamber 277 of the second incubator 277 is maintained at 30±1° C. At this time, it may be detected whether or not the second dry analysis chip 203 has certainly been inserted into the chip chamber 277 of the second incubator 208. If the second dry analysis chip 203 is inserted into the second incubator 208, a measurement of ionic activity is made by the second measurement means 212. After the measurement, the chip chamber 277 is moved upward, and the measured dry analysis chip 203 is discarded into the chip-discarding bore 256 of the first incubator 207 through the chip chamber 255 of the first incubator 207 by the insertion member 241. The result of measurement is output, and the reference-solution nozzle tip 221 and the electrolytic-inspection-matter nozzle tip 221 that have been used are removed from the dropping nozzle 301a with the nozzle-tip removing section 220. The removed nozzle tips 221 are dropped and discarded and the process is ended.

The insertion of the first dry analysis chip 202 into the first incubator and the measurement of the first dry analysis chip 202 can be sequentially performed by the number of chip chambers 255. However, since the second incubator 208 has only one chip chamber 277, dropping of inspection matter with respect to the second dry analysis chip 203 is not performed during the time that the previous dry analysis chip 203 is being measured. On the other hand, dropping of inspection matter with respect to the first dry analysis chip 202 is performed and the first dry analysis chip 202 is inserted into the first incubator 207 through the passage 236 of the distributing section 236. In addition, the second incubator 208 and the second measurement means 212 may be provided so that they are moved up and down according to movement of the distributing section 206.

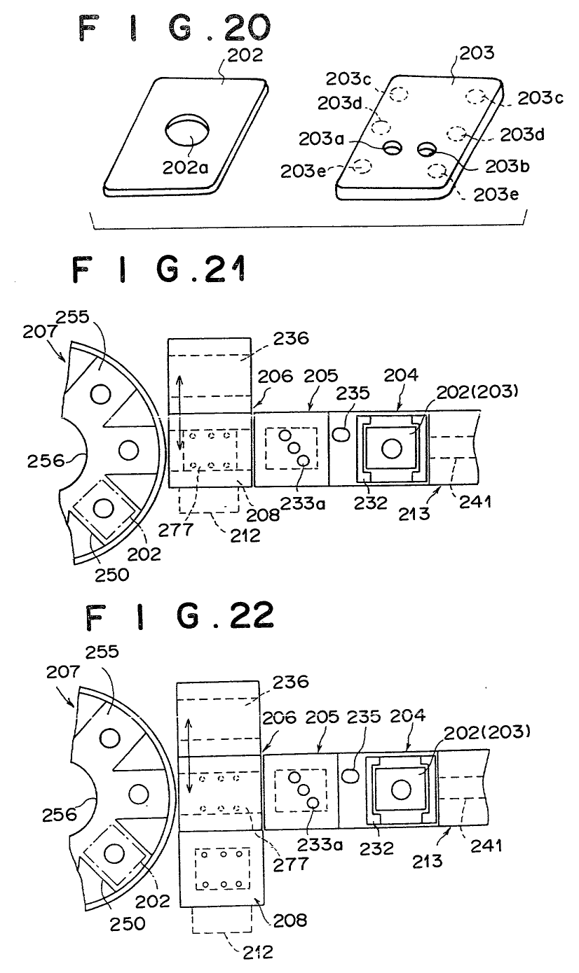

FIG. 21 illustrates the distributing section of a biochemical analysis apparatus constructed according to a third embodiment of the present invention. In the third embodiment, the chip chamber is movable in a horizontal direction.

The distributing section 206 of the third embodiment, disposed between a dropping section 205 and a first incubator 207, has a passage 236 for conveying the first dry analysis chip 203 to the first incubator 207. The passage 236 is formed parallel to the chip chamber 277 of a second incubator 208 in a horizontal direction. The second incubator 208 and second measurement means 212 are horizontally moved along with horizontal movement of the chip chamber 277.

In the case where the second dry analysis chip 203 has not been inserted in the chip chamber 277 when the first dry analysis chip 202 is conveyed from the dropping section 205 to the first incubator 207, the distributing section 206 is moved so that the chip chamber 277 or passage 236 is connected with the conveying path of the dry analysis chip. Therefore, the first dry analysis chip 202 is inserted into the first incubator 207 through the distributing section 206 and is measured. When the second dry analysis chip 203 is conveyed from the dropping section 205, the chip chamber 277 of the distributing section 206 is moved so that the second dry analysis chip 203 is inserted and held in the chip chamber 277. Thereafter, the chip chamber 277 is moved sidewise along with the second incubator 208 and the second measurement means 212, and the passage 236 is moved so that it is connected with the conveying path. In this stage, the second dry analysis chip 203 is measured. On the other hand, the first dry analysis chip 202, following the second dry analysis chip 203, is inserted into the first incubator 7 through the passage 236 of the distributing section 206 moved, and the measurement is performed at the same time.

FIG. 22 illustrates the distributing section of a biochemical analysis apparatus constructed according to a fourth embodiment of the present invention. In the fourth embodiment, the chip chamber is movable in a horizontal direction, but the second incubator 208 and the second measurement means 212 are fixedly disposed.

The distributing section 206 of the fourth embodiment, disposed between a dropping section 205 and a first incubator 207, has a passage 236 for conveying the first dry analysis chip 203 to the first incubator 207. The passage 236 is formed parallel to the chip chamber 277 of a second incubator 208 in a horizontal direction. The passage 236 and the chip chamber 277 is selectively switched so that they are connected with the conveying path. The main body of the second incubator 208 and second measurement means 212 are fixedly disposed on the side of the distributing section 206, and the chip chamber 277 of the second incubator 208 is movable to the distributing section 206.

In the case where the second dry analysis chip 203 has not been inserted in the chip chamber 277 when the first dry analysis chip 202 is conveyed from the dropping section 205 to the first incubator 207, the distributing section 206 is moved so that the chip chamber 277 or passage 236 is connected with the conveying path. Therefore, the first dry analysis chip 202 is inserted into the first incubator 207 through the distributing section 206 and is measured. When the second dry analysis chip 203 is conveyed from the dropping section 205, the chip chamber 277 of the distributing section 206 is moved so that the second dry analysis chip 203 is inserted and held in the chip chamber 277. Thereafter, the chip chamber 277 is moved to the second incubator 208 and the second measurement means 212. In this stage, the second dry analysis chip 203 is measured. On the other hand, the first dry analysis chip 202, following the second dry analysis chip 203, is inserted into the first incubator 7 through the passage 236 of the distributing section 206, and the measurement is performed at the same time.

In the aforementioned embodiments, when the second dry analysis chip 203 has not been inserted into the chip chamber 277 of the second incubator 208 of the distributing section 206, the chip chamber 277 is connected with the conveying path so that the first dry analysis chip 202 is conveyed to the first incubator 207 through the chip chamber 277. However, in this state, the passage 236 may be connected with the conveying path so that the first dry analysis chip 202 is always passed through the passage 236.

Thus, in the aforementioned embodiments, the bar code of the dry analysis chip 202 or 203 is read and the type is identified. According to the type, dropping of inspection matter is performed. Also, the second dry analysis chip 203 is inserted into the chip chamber 277 of the second incubator 208 of the distributing section 206, while the first dry analysis chip 202 is inserted from the distributing section 206 into the first incubator 207. The first and second dry analysis chips 202, 203 are incubated at their respective incubation temperatures, and the calorimetric measurement and potential difference measurement are made by the first measurement means 211 and the second measurement means 212 to detect the substance density and ionic activity. In addition, the substance density measurement and the ionic activity measurement can be simultaneously made by the first measurement means 211 and the second measurement means 212, so the dry analysis chips 202, 203 can be efficiently processed with compact construction.

The number of dry analysis chips 202, 203 that are housed in the first and second incubators 207, 208 in the aforementioned embodiments is arbitrary. However, it is preferable from the actual ratio of measurement to hold six analysis chips in the first incubator 207 and one analysis chip in the second incubator 208.

In the aforementioned embodiments, the first and second dry analysis chips 202, 203 are incubated at different temperatures by the first and second incubators 207, 208. However, they may be incubated at the same temperature by the first and second incubators 207, 208.

While the present invention has been described with reference to the preferred embodiments thereof, the invention is not to be limited to the details given herein, but may be modified within the scope of the invention hereinafter claimed.

What is claimed is:

1. A biochemical analysis apparatus comprising:
first and second dry analysis chips, different in method of measurement, which have inspection matter dropped thereon;
a first incubator for housing said first dry analysis chip and incubating said first dry analysis chip at a first predetermined temperature;
first measurement means provided in said first incubator;
a second incubator for housing said second dry analysis chip and incubating said second dry analysis chip at a second predetermined temperature;
second measurement means provided in said second incubator; and
conveyance means for conveying said first and second dry analysis chips to said first and second incubators through first and second conveying paths.

2. The biochemical analysis apparatus as set forth in claim 1, wherein said conveyance means comprises
a first conveying member for conveying said first and second dry analysis chips to a distributing section and also conveying said first dry analysis chip from said distributing section to said first incubator; and
a second conveying member for conveying said second dry analysis chip from said distributing section to said second incubator.

3. The biochemical analysis apparatus as set forth in claim 2, wherein said distributing section comprises
a first guide pair, which projects from a conveying surface, for guiding said first dry analysis chip to said first incubator; and
a second guide pair, which projects from said conveying surface, for guiding said second dry analysis chip to said second incubator.

4. The biochemical analysis apparatus as set forth in claim 1, wherein said first and second dry analysis chips after measurement are conveyed beyond said first and second incubators and are discarded.

5. The biochemical analysis apparatus as set forth in claim 2, wherein said first and second dry analysis chips after measurement are conveyed beyond said first and second incubators and are discarded.

6. The biochemical analysis apparatus as set forth in claim 3, wherein said first and second dry analysis chips after measurement are conveyed beyond said first and second incubators and are discarded.

7. The biochemical analysis apparatus as set forth in any one of claims 1-6, wherein
said first and second dry analysis chips are each provided with a bar code that indicates its type;
said bar code is read before the dropping of said inspection matter; and
according to said type, said dropping, conveyance, incubation, and measurement are performed.

8. The biochemical analysis apparatus as set forth in claim 1, wherein
said first dry analysis chip is a colorimetric type dry analysis chip for measuring a substance density of a predetermined biochemical substance contained in said inspection matter by color reaction;
said second dry analysis chip is an electrolytic type dry analysis chip for measuring ionic activity of said inspection matter;
said first measurement means comprises a color-reaction measuring section for measuring a change in optical density by color reaction between said predetermined biochemical substance and a reagent;
said second measurement means comprises a potential-difference measuring section equipped with probes for measuring a potential difference between said inspection matter and a reference solution which corresponds to said ionic activity;
said first incubator has a plurality of chip chambers, and a measurement is made in sequence with said first measurement means; and
said second incubator has a single chip chamber, and a measurement is made with said second measurement means.

9. A biochemical analysis apparatus comprising:
first and second dry analysis chips differing in method of measurement;
a dropping section for dropping inspection matter to said first and second dry analysis chips;
a first incubator for housing said first dry analysis chip which has the inspection matter dropped thereon and then incubating said first dry analysis chip at a first predetermined temperature;
first measurement means provided in said first incubator;
a second incubator for housing said second dry analysis chip which has the inspection matter dropped thereon and then incubating said second dry analysis chip at a second predetermined temperature;
second measurement means provided in said second incubator; and
a distributing section disposed between said dropping section and said first incubator;
wherein a passage for conveying said first dry analysis chip to said first incubator, and a chip chamber of said second incubator, are provided in said distributing section so that said passage and said chip chamber can be switched between them;
and wherein said second incubator and said second measurement means are disposed in said distributing section.

10. The biochemical analysis apparatus as set forth in claim 9, wherein said passage and said chip chamber in said distributing section are provided parallel to each other in a vertical direction with respect to a conveying path and are movable up and down, depending on dry analysis chip type.

11. The biochemical analysis apparatus as set forth in claim 9, wherein said passage and said chip chamber in said distributing section are provided parallel to each other in a lateral direction with respect to a conveying path and are movable in said lateral direction, depending on dry analysis chip type.

12. The biochemical analysis apparatus as set forth in claim 10, wherein said second incubator is movable integrally with movement of said passage and said chip chamber in said distributing section.

13. The biochemical analysis apparatus as set forth in claim 11, wherein said second incubator is movable integrally with movement of said passage and said chip chamber in said distributing section.

14. The biochemical analysis apparatus as set forth in claim 10, wherein said second incubator is fixedly disposed and said chip chamber with said second dry analysis chip housed therein is movable with respect to said second incubator.

15. The biochemical analysis apparatus as set forth in claim 11, wherein said second incubator is fixedly disposed and said chip chamber with said second dry analysis chip housed therein is movable with respect to said second incubator.

16. The biochemical analysis apparatus as set forth in any one of claims 11-15, wherein
said first and second dry analysis chips are each provided with a bar code that represents type;
said bar code is read before dropping of said inspection matter; and
according to said type, said dropping, conveyance, incubation, and measurement are performed.

17. The biochemical analysis apparatus as set forth in claim 9, wherein
said first dry analysis chip is a calorimetric type dry analysis chip for measuring a substance density of a predetermined biochemical substance contained in said inspection matter by color reaction;
said second dry analysis chip is an electrolytic type dry analysis chip for measuring ionic activity of said inspection matter;
said first measurement means comprises a color-reaction measuring section for measuring a change in optical density by color reaction between said predetermined biochemical substance and a reagent;
said second measurement means comprises a potential-difference measuring section equipped with probes for measuring a potential difference between said inspection matter and a reference solution which corresponds to said ionic activity;
said first incubator has a plurality of chip chambers, and a measurement is made in sequence with said first measurement means; and
said second incubator has a single chip chamber, and when said second dry analysis chip is inserted into said single chip chamber and moved, the probes of said second measurement means are connected electrically with said second dry analysis chip.
